(19) United States
(12) Patent Application Publication
(10) Pub. No.: US 2025/0275348 A1
(43) Pub. Date: Aug. 28, 2025

(54) Light-Emitting Device, Stacked Light-Emitting Device, and Display Substrate

(71) Applicant: BOE TECHNOLOGY GROUP CO., LTD., Chaoyang District, Beijing (CN)

(72) Inventors: Lei Chen, Beijing (CN); Dan Wang, Beijing (CN); Rongrong Gao, Beijing (CN)

(21) Appl. No.: 18/858,014

(22) PCT Filed: Jul. 4, 2023
(86) PCT No.: PCT/CN2023/105761

§ 371 Date: Oct. 18, 2024

(30) Foreign Application Priority Data
Jul. 6, 2022 (CN) 202210788076.X

(51) Int. Cl. H10K 50/12; H10K 50/19; H10K 59/80; H10K 85/60; H10K 101/00; H10K 101/30

(52) U.S. Cl. CPC H10K 50/12; H10K 50/19; H10K 59/873; H10K 85/615; H10K 85/6574; H10K 2101/30; H10K 2101/90

(57) ABSTRACT

A stacked light-emitting device includes a first electrode, a second electrode, at least two light-emitting units, and at least one stacked connection layer. A stacked connection layer is between every two adjacent light-emitting units and includes an N-type charge generation layer and a P-type charge generation layer. The N-type charge generation layer is of a doped binary structure including a first host material and a first guest material; and the P-type charge generation layer is of a doped binary structure including a second host material and a second guest material. An absolute value of difference between HOMO energy level of the second host material and HOMO energy level of the first host material is greater than 0.3 electron volts; and an absolute value of difference between LUMO energy level of the second host material and LUMO energy level of the first host material is greater than 0.1 electron volts.

### CROSS-REFERENCE TO RELATED APPLICATIONS

This application is the United States national phase of International Patent Application No. PCT/CN2023/105761 filed Jul. 4, 2023, and claims priority to Chinese Patent Application No. 202210788076.X, filed Jul. 6, 2022, the disclosures of which are hereby incorporated by reference in their entireties.

### BACKGROUND OF THE INVENTION

### Field of the Invention

The present disclosure relates to the field of display technologies, and in particular, to an organic matter, a light-emitting device, a stacked light-emitting device, a display substrate, and a display apparatus.

### Description of Related Art

In the field of display technologies, organic light-emitting diode (OLED) display apparatuses have been widely used in a variety of fields, such as flat panel displays, flexible displays, in-vehicle displays, and solid-state lighting, owing to their advantages of wide color gamut, high contrast, energy efficiency, and foldability.

### SUMMARY OF THE INVENTION

In an aspect, a stacked light-emitting device is provided, which includes a first electrode, a second electrode, at least two light-emitting units, and at least one stacked connection layer. The at least two light-emitting units are stacked between the first electrode and the second electrode. A stacked connection layer in the at least one stacked connection layer is disposed between every two adjacent light-emitting units in the at least two light-emitting units, and the stacked connection layer includes an N-type charge generation layer and a P-type charge generation layer disposed in a stack.

The N-type charge generation layer is of a doped binary structure including a first host material and a first guest material; the P-type charge generation layer is of a doped binary structure including a second host material and a second guest material; an absolute value of a difference between a highest occupied molecular orbital energy level of the second host material and a highest occupied molecular orbital energy level of the first host material is greater than 0.3 electron volts; and an absolute value of a difference between a lowest unoccupied molecular orbital energy level of the second host material and a lowest unoccupied molecular orbital energy level of the first host material is greater than 0.1 electron volts.

In some embodiments, the first guest material includes at least one of a metal or an organic matter; in a case where the first guest material is the metal, an absolute value of a difference between a work function of the first guest material and the lowest unoccupied molecular orbital energy level of the first host material is less than 1.0 electron volts; in a case where the first guest material is the organic matter, an absolute value of a difference between a highest occupied molecular orbital energy level of the first guest material and the lowest unoccupied molecular orbital energy level of the first host material is less than 1.0 electron volts.

In some embodiments, an absolute value of a difference between a lowest unoccupied molecular orbital energy level of the second guest material and the highest occupied molecular orbital energy level of the second host material is less than 0.5 electron volts.

In some embodiments, a structure of the first host material has a conjugated fragment; and the conjugated fragment has at least two benzene rings, and all of the benzene rings in the conjugated fragment are of a π-π conjugated structure.

In some embodiments, at least one substituent on the conjugated fragment has a phosphorus oxygen group.

In some embodiments, the first host material has a structure shown in formula (I):

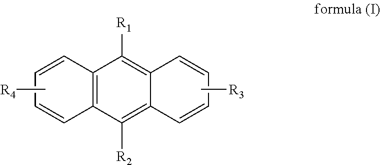

where R₁, R₂, R₃ and R₄ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, a substituted or unsubstituted C₆ to C₃₀ aryloxy group, and a structure shown in formula (II);
where at least one of the R₁, the R₂, the R₃ and the R₄ has the structure shown in the formula (II):

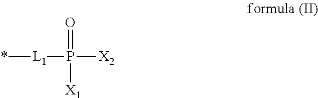

where * indicates a site connected to a carbon atom; L₁ is selected from any one of: single bond, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group; and X₁ are X₂ each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group.

In some embodiments, at least one of the R₃ and the R₄ has the structure shown in the formula (II).

In some embodiments, both R₃ and R₄ have the structure shown in the formula (II).

In some embodiments, [the first host material has a structure shown in any one of formula (1-1) to formula (1-10):

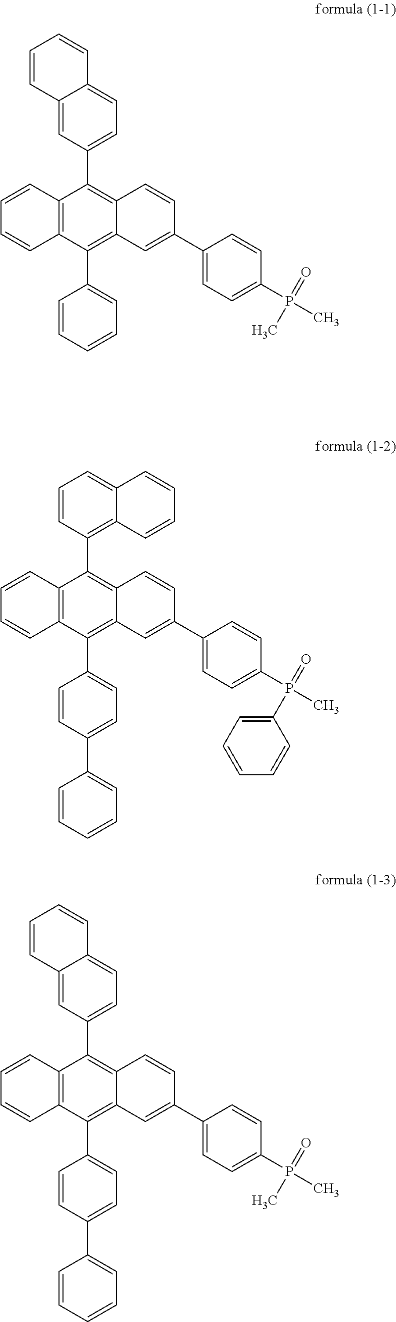 [SMILES: CP(C)(=O)c1ccc(-c2ccc3c(-c4ccc5ccccc5c4)c4ccccc4c(-c4ccccc4)c3c2)cc1 ; CP(=O)(c1ccccc1)c1ccc(-c2ccc3c(-c4cccc5ccccc45)c4ccccc4c(-c4ccc(-c5ccccc5)cc4)c3c2)cc1 ; CP(C)(=O)c1ccc(-c2ccc3c(-c4ccc5ccccc5c4)c4ccccc4c(-c4ccc(-c5ccccc5)cc4)c3c2)cc1]

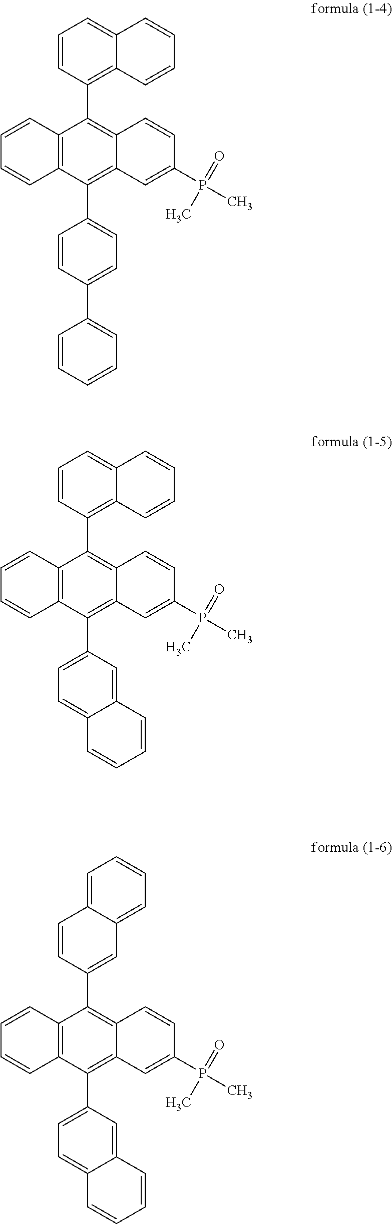 [SMILES: CP(C)(=O)c1ccc2c(-c3cccc4ccccc34)c3ccccc3c(-c3ccc(-c4ccccc4)cc3)c2c1 ; CP(C)(=O)c1ccc2c(-c3cccc4ccccc34)c3ccccc3c(-c3ccc4ccccc4c3)c2c1 ; CP(C)(=O)c1ccc2c(-c3ccc4ccccc4c3)c3ccccc3c(-c3ccc4ccccc4c3)c2c1]

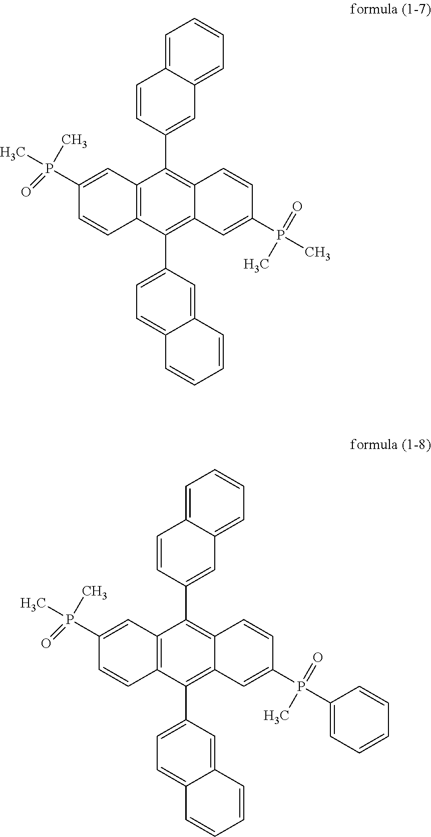

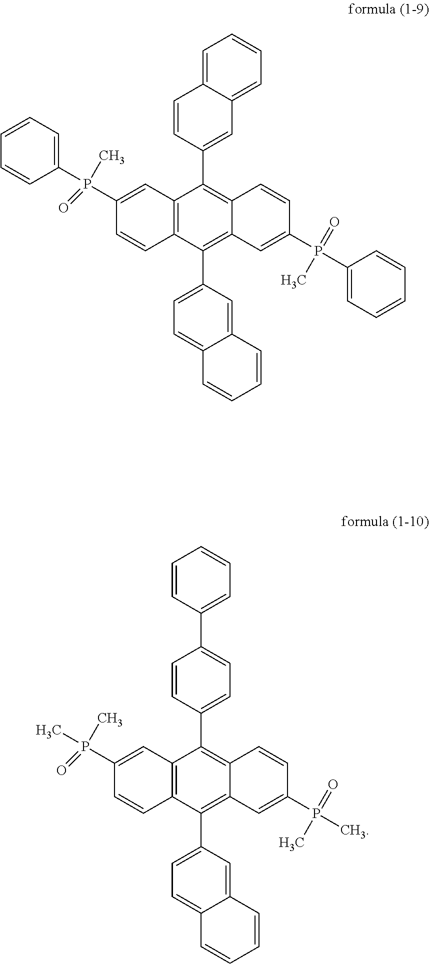 [SMILES: CP(=O)(c1ccccc1)c1ccc2c(-c3ccc4ccccc4c3)c3cc(P(C)(=O)c4ccccc4)ccc3c(-c3ccc4ccccc4c3)c2c1 ; CP(C)(=O)c1ccc2c(-c3ccc4ccccc4c3)c3cc(P(C)(C)=O)ccc3c(-c3ccc(-c4ccccc4)cc3)c2c1]

In some embodiments, the light-emitting units each include a light-emitting layer, and the light-emitting layer is of a doped binary structure including a third host material and a third guest material; a structure of the third host material has a conjugated fragment; and the conjugated fragment has at least two benzene rings, and all of the benzene rings in the conjugated fragment are of a π-π conjugated structure.

In some embodiments, the third host material has a structure shown in formula (III):

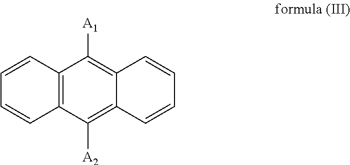

where A₁ and A₂ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group.

In some embodiments, the third host material has a structure shown in any one of formula (3-1) to formula (3-12):

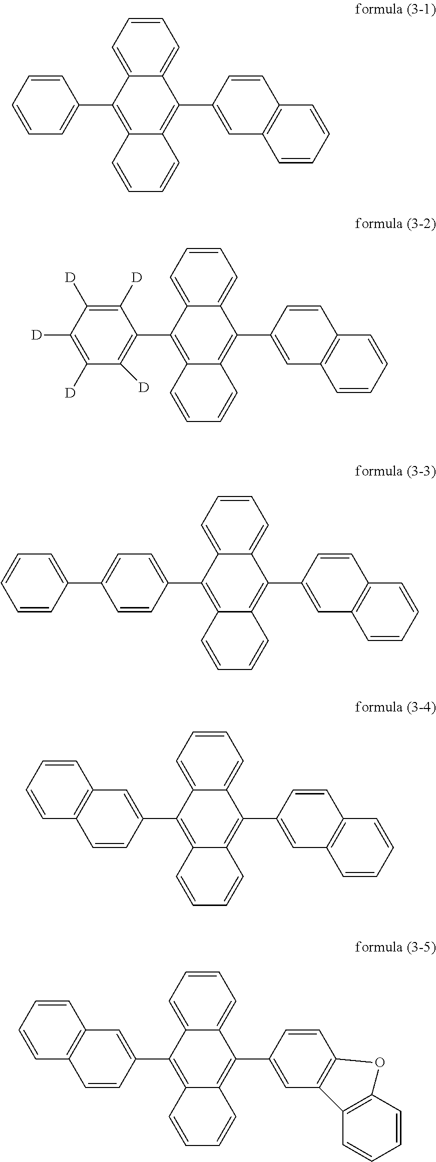

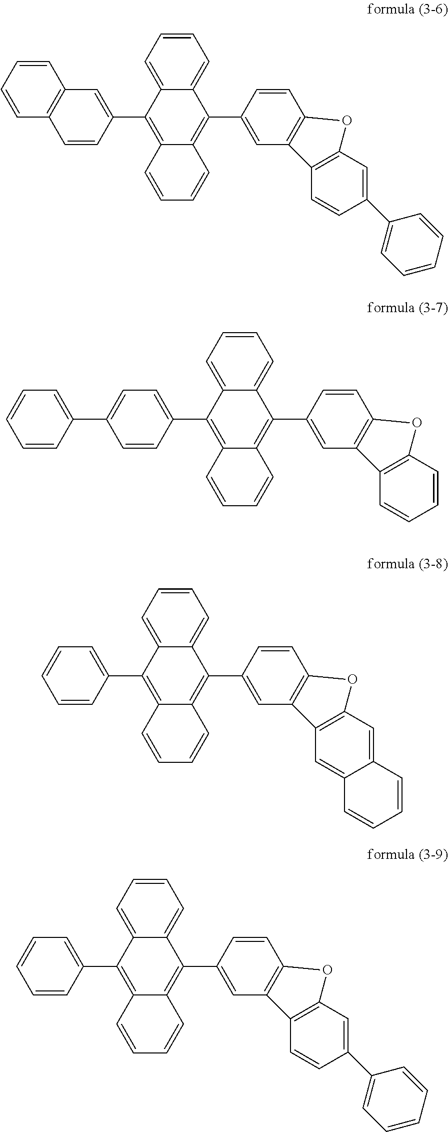 [SMILES: c1ccc(-c2ccc3c(c2)oc2ccc(-c4c5ccccc5c(-c5ccc6ccccc6c5)c5ccccc45)cc23)cc1 ; c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc3c(c2)oc2ccc(-c4c5ccccc5c(-c5ccccc5)c5ccccc45)cc23)cc1]

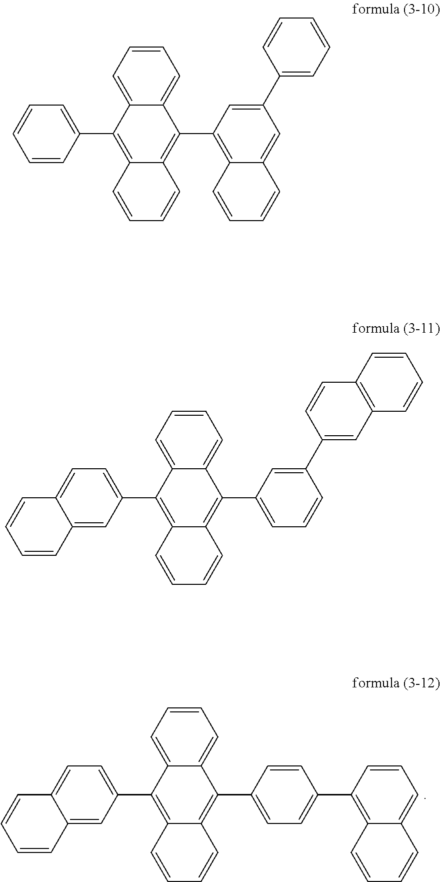

In another aspect, a stacked light-emitting device is provided, which includes: a first electrode, a second electrode, at least two light-emitting units, and at least one stacked connection layer. The at least two light-emitting units are stacked between the first electrode and the second electrode; the light-emitting units each including a light-emitting layer. A stacked connection layer in the at least one stacked connection layer is disposed between every two adjacent light-emitting units in the at least two light-emitting units, and the stacked connection layer includes an N-type charge generation layer and a P-type charge generation layer disposed in a stack.

At least three film layers in the stacked light-emitting device each include a material having a conjugated fragment, and the at least three film layers include at least one of the light-emitting layer and the N-type charge generation layer.

In some embodiments, the conjugated fragment has at least two benzene rings, and all of the benzene rings in the conjugated fragment have a π-π conjugated structure.

In some embodiments, of the at least three film layers in the stacked light-emitting device, at least one material having a conjugated fragment has a phosphorus oxygen group in at least one substituent on the conjugated fragment.

In some embodiments, of the at least three film layers in the stacked light-emitting device, at least two film layers have different conjugated fragments.

In some embodiments, the at least three film layers in the stacked light-emitting device include at least two light-emitting layers and an N-type charge generation layer located between the at least two light-emitting layers.

In some embodiments, all light-emitting layers of the stacked light-emitting device include at least two light-emitting layers of a same material.

In some embodiments, the light-emitting layer includes a doped binary structure including a third host material and a third guest material; and a structure of the third host material has the conjugated fragment.

In some embodiments, the third host material has a structure shown in formula (III):

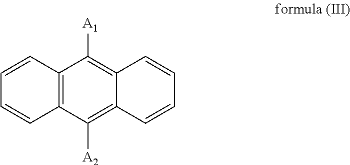

where A₁ and A₂ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group.

In some embodiments, the third host material has a structure shown in any one of formula (3-1) to formula (3-12):

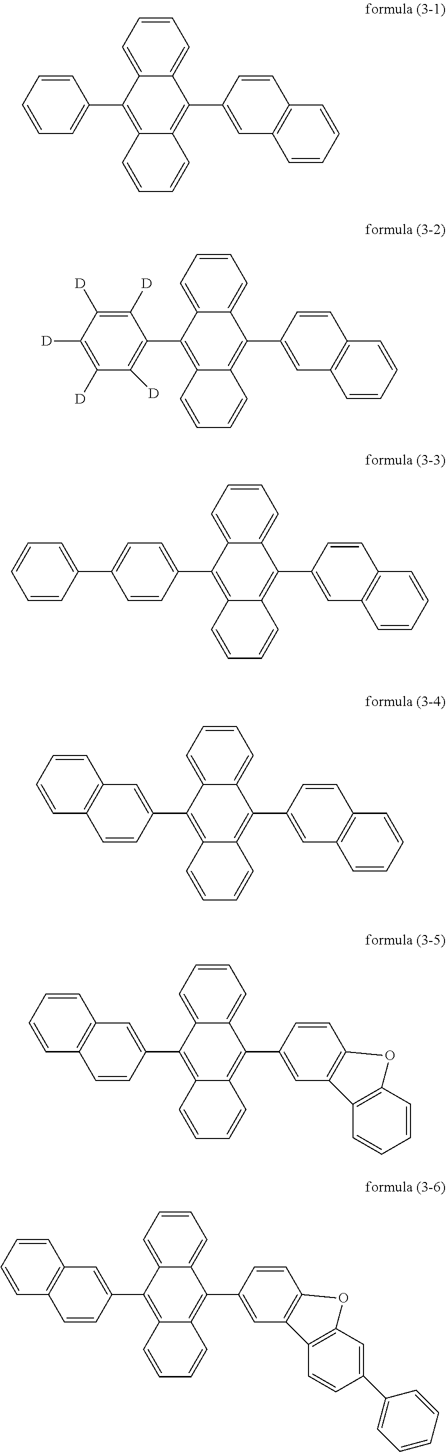

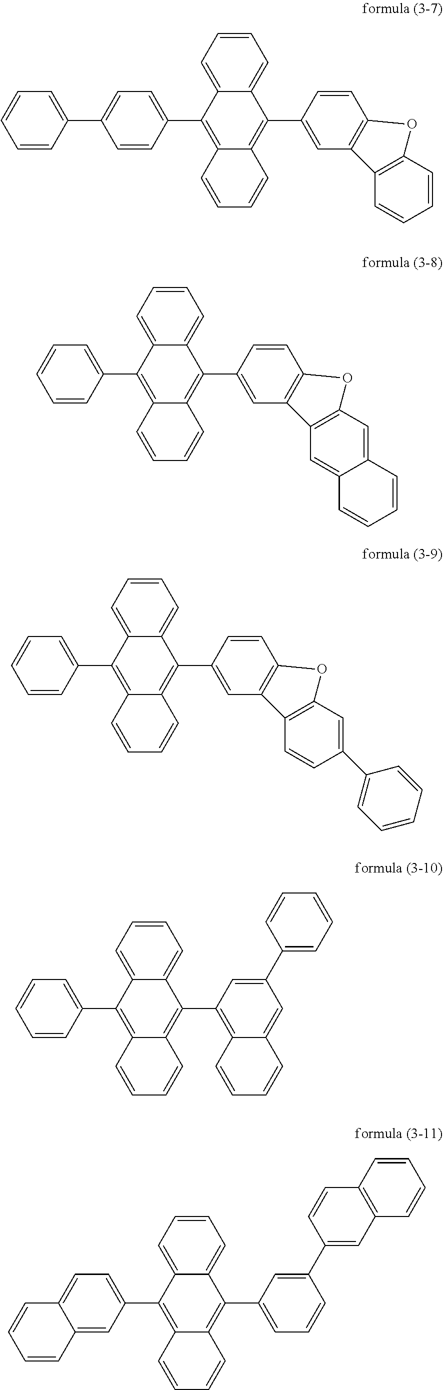

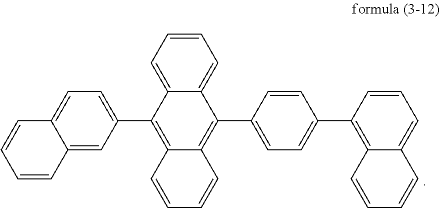 [SMILES: c1ccc2cc(-c3c4ccccc4c(-c4ccc(-c5cccc6ccccc56)cc4)c4ccccc34)ccc2c1]

In some embodiments, the N-type charge generation layer includes a doped binary structure including a first host material and a first guest material; and a structure of the first host material has the conjugated fragment.

In some embodiments, the P-type charge generation layer is of a doped binary structure including a second host material and a second guest material; an absolute value of a difference between a highest occupied molecular orbital energy level of the second host material and a highest occupied molecular orbital energy level of the first host material is greater than 0.3 electron volts; and an absolute value of a difference between a lowest unoccupied molecular orbital energy level of the second host material and a lowest unoccupied molecular orbital energy level of the first host material is greater than 0.1 electron volts.

In some embodiments, the first guest material includes at least one of a metal or an organic matter. In a case where the first guest material includes the metal, an absolute value of a difference between a work function of the metal included in the first guest material and the lowest unoccupied molecular orbital energy level of the first host material is less than 1.0 electron volts; in a case where the first guest material includes the organic matter, an absolute value of a difference between a highest occupied molecular orbital energy level of the organic matter included in the first guest material and the lowest unoccupied molecular orbital energy level of the first host material is less than 1.0 electron volts.

In some embodiments, an absolute value of a difference between a lowest unoccupied molecular orbital energy level of the second guest material and the highest occupied molecular orbital energy level of the second host material is less than 0.5 electron volts.

In some embodiments, the first host material has a structure shown in formula (1):

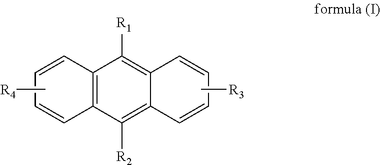

where R₁, R₂, R₃ and R₄ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, a substituted or unsubstituted C₆ to C₃₀ aryloxy group, and a structure shown in formula (II), where at least one of the R₁, the R₂, the R₃ and the R₄ has the structure shown in the formula (II):

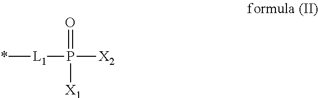

where * indicates a site connected to a carbon atom;
L₁ is selected from any one of: single bond, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C to Co aralkyl group, a substituted or unsubstituted C₁ to C₂₀ alkoxy group, and a substituted or unsubstituted C₆ to C₆₀ aryloxy group; and
X₁ are X₂ each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₃₀ aryloxy group.

In some embodiments, the first host material has a structure shown in any one of formula (1-1) to formula (1-10):

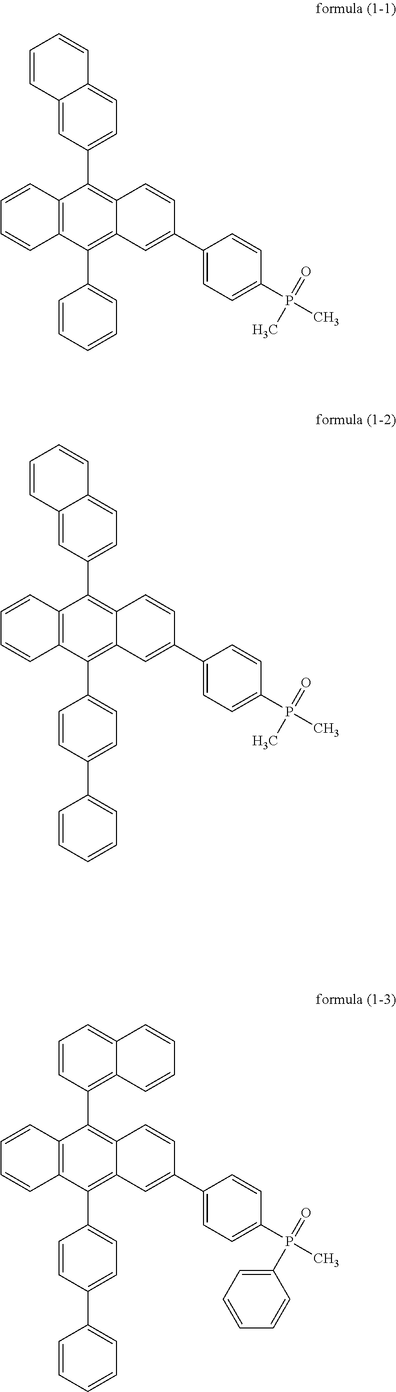 [SMILES: CP(C)(=O)c1ccc(-c2ccc3c(-c4ccc5ccccc5c4)c4ccccc4c(-c4ccccc4)c3c2)cc1 ; CP(C)(=O)c1ccc(-c2ccc3c(-c4ccc5ccccc5c4)c4ccccc4c(-c4ccc(-c5ccccc5)cc4)c3c2)cc1 ; CP(=O)(c1ccccc1)c1ccc(-c2ccc3c(-c4cccc5ccccc45)c4ccccc4c(-c4ccc(-c5ccccc5)cc4)c3c2)cc1]

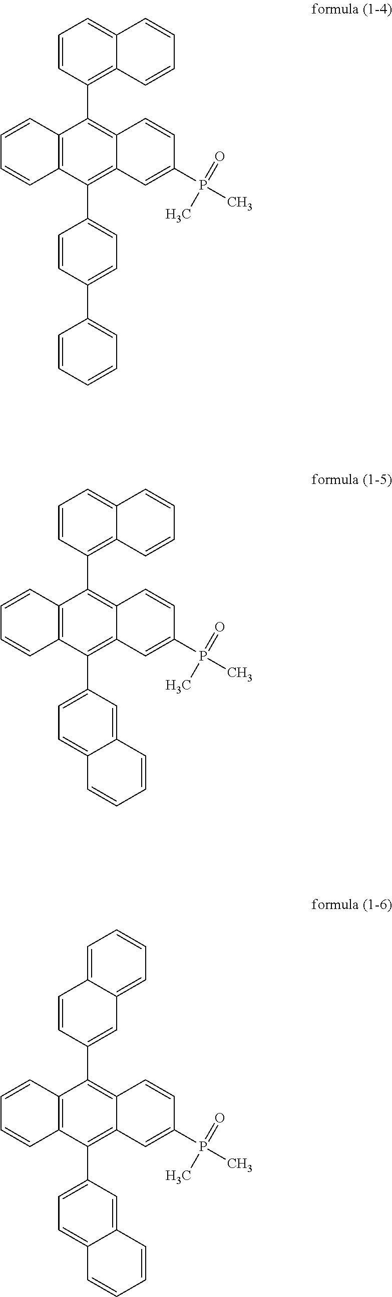 [SMILES: CP(C)(=O)c1ccc2c(-c3cccc4ccccc34)c3ccccc3c(-c3ccc(-c4ccccc4)cc3)c2c1 ; CP(C)(=O)c1ccc2c(-c3cccc4ccccc34)c3ccccc3c(-c3ccc4ccccc4c3)c2c1 ; CP(C)(=O)c1ccc2c(-c3ccc4ccccc4c3)c3ccccc3c(-c3ccc4ccccc4c3)c2c1]

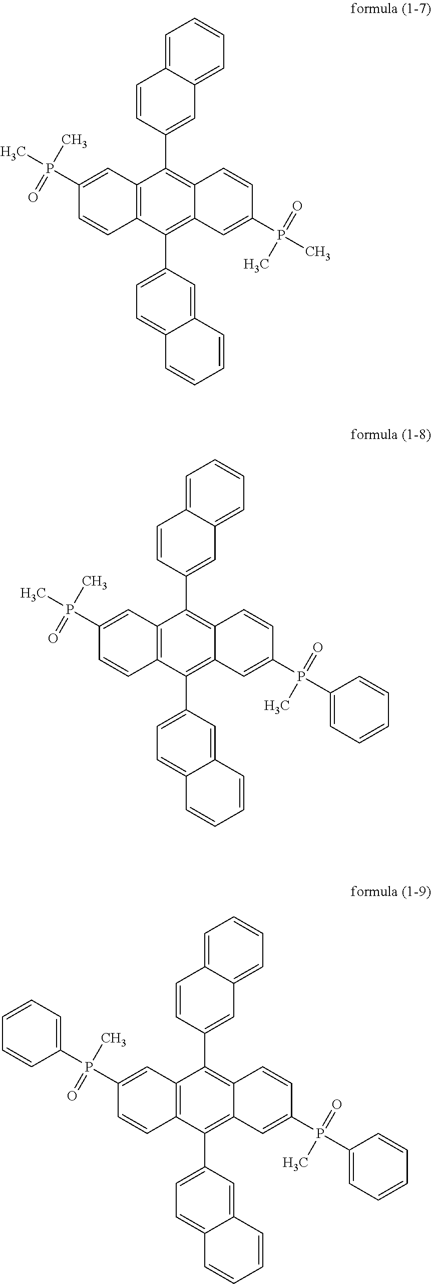

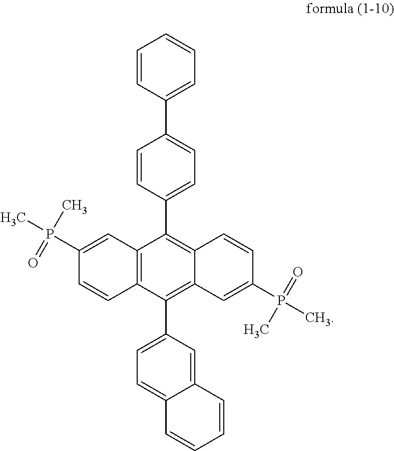 [SMILES: CP(C)(=O)c1ccc2c(-c3ccc4ccccc4c3)c3cc(P(C)(C)=O)ccc3c(-c3ccc(-c4ccccc4)cc3)c2c1]

In yet another aspect, a light-emitting device is provided, which includes: a first electrode, a second electrode, and at least one light-emitting unit. The at least one light-emitting unit is disposed between the first electrode and the second electrode; the light-emitting units each including a light-emitting layer.

The light-emitting layer is of a doped binary structure including a third host material and a third guest material; a structure of the third host material has a conjugated fragment; and the conjugated fragment has at least two benzene rings, and all of the benzene rings in the conjugated fragment are of a π-π conjugated structure.

In some embodiments, the third host material has a structure shown in formula (III):

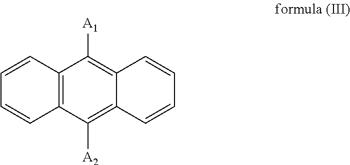

where A₁ and A₂ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group.

In some embodiments, the third host material has a structure shown in any one of formula (3-1) to formula (3-12):

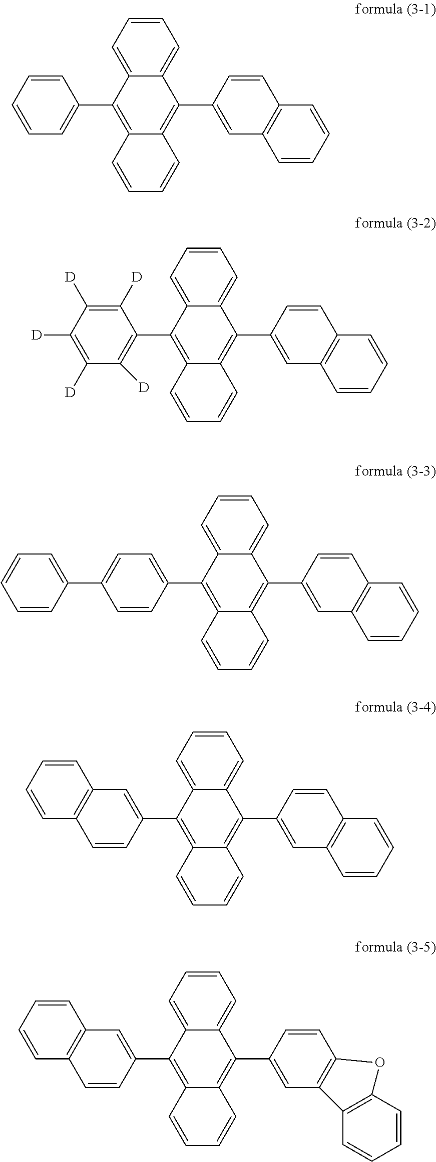

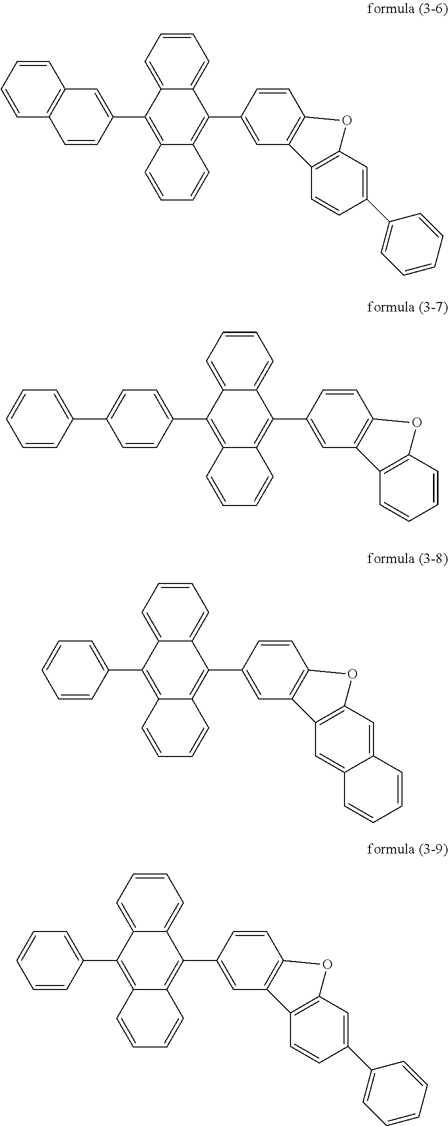 [SMILES: c1ccc(-c2ccc3c(c2)oc2ccc(-c4c5ccccc5c(-c5ccc6ccccc6c5)c5ccccc45)cc23)cc1 ; c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc3c(c2)oc2ccc(-c4c5ccccc5c(-c5ccccc5)c5ccccc45)cc23)cc1]

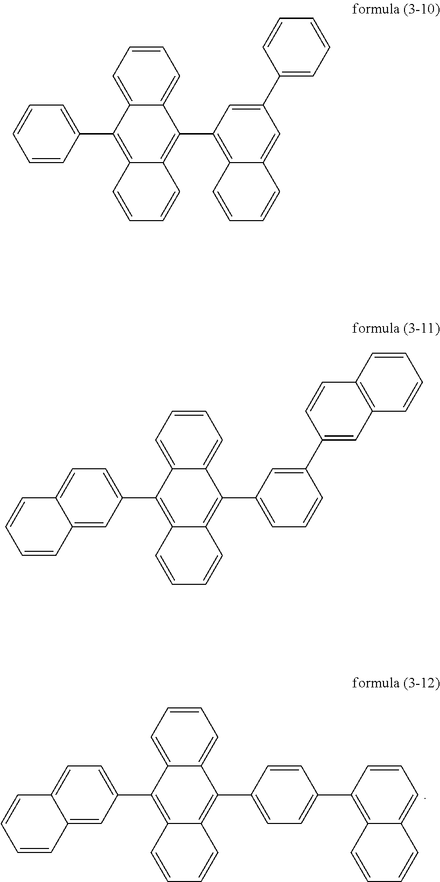

In still yet another aspect, a display substrate is provided, which includes: a substrate, a plurality of light-emitting devices, a plurality of pixel driving circuits, and an encapsulation layer. At least one of the plurality of light-emitting devices is the stacked light-emitting device according to any one of the above embodiments or the light-emitting device according to the above embodiments. The plurality of pixel driving circuits is used for driving the plurality of light-emitting devices to emit light. The encapsulation layer is used for encapsulating the plurality of light-emitting devices and the plurality of pixel driving circuits.

In still yet another aspect, a display apparatus is provided, which includes the display substrate according to the above embodiments.

In still yet another aspect, an organic matter is provided, which has a structure shown in formula (III):

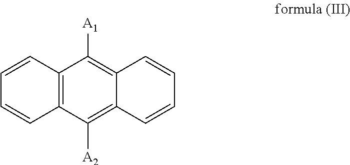

where A₁ and A₂ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group.

In some embodiments, the organic matter has a structure shown in any one of formula (3-1) to formula (3-12):

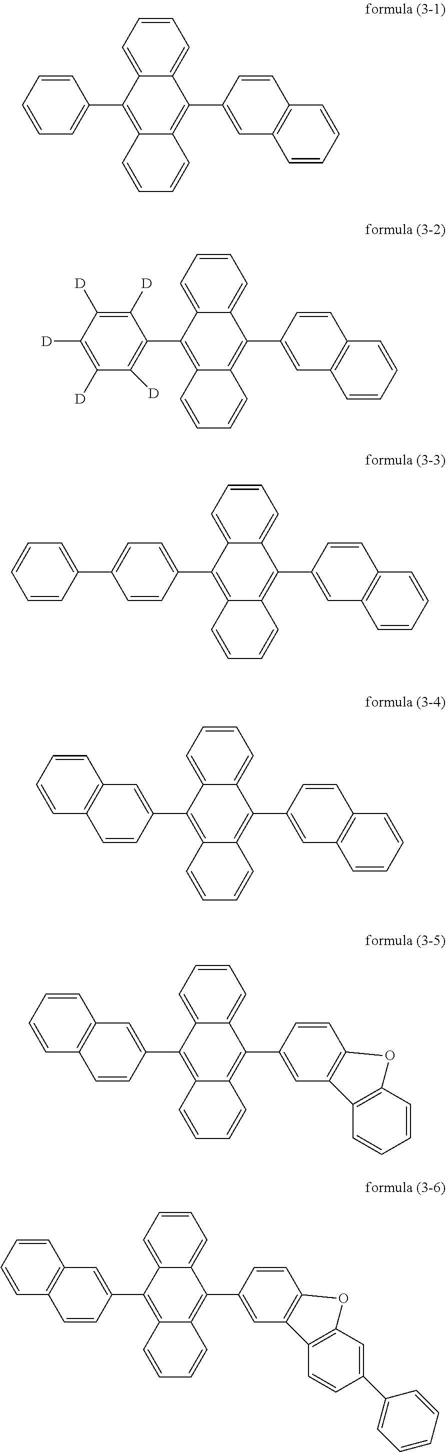

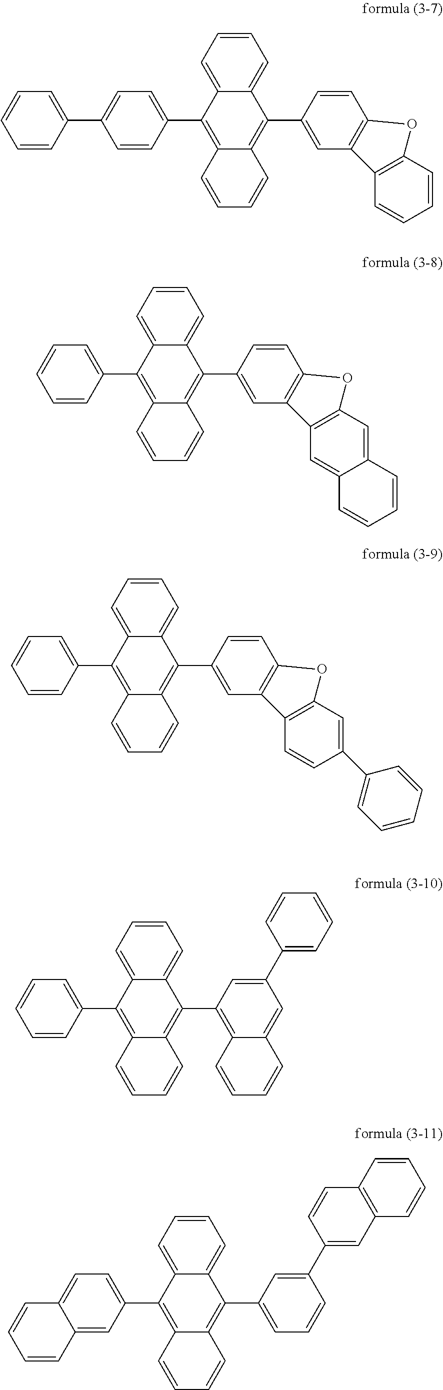

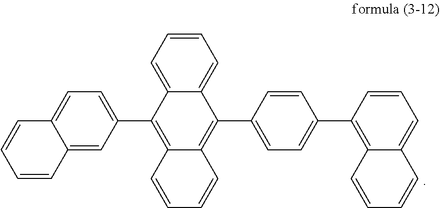 [SMILES: c1ccc2cc(-c3c4ccccc4c(-c4ccc(-c5cccc6ccccc56)cc4)c4ccccc34)ccc2c1]

### BRIEF DESCRIPTION OF THE DRAWINGS

In order to describe technical solutions in the present disclosure more clearly, the accompanying drawings to be used in some embodiments of the present disclosure will be introduced briefly. Obviously, the accompanying drawings to be described below are merely drawings of some embodiments of the present disclosure, and a person of ordinary skill in the art can obtain other drawings according to those drawings. In addition, the accompanying drawings in the following description may be regarded as schematic diagrams, but are not limitations on actual sizes of products, actual processes of methods and actual timings of signals involved in the embodiments of the present disclosure.

FIG. 1 is a structural diagram of a display apparatus, in accordance with some embodiments;

FIG. 2 is a structural diagram of a display module, in accordance with some embodiments;

FIG. 3 is a structural diagram of a display substrate, in accordance with some embodiments;

FIG. 4 is a structural diagram of a light-emitting device in the related art;

FIG. 5 is a structural diagram of a stacked light-emitting device in the related art;

FIG. 6 is a structural diagram of a stacked light-emitting device, in accordance with some embodiments; and

FIG. 7 is a structural diagram of a light-emitting device, in accordance with some embodiments.

### DESCRIPTION OF THE INVENTION

The technical solutions in some embodiments of the present disclosure will be described clearly and completely with reference to the accompanying drawings; obviously, the described embodiments are merely some but not all embodiments of the present disclosure. All other embodiments obtained by a person of ordinary skill in the art based on embodiments of the present disclosure shall be included in the protection scope of the present disclosure.

Unless the context requires otherwise, throughout the specification and the claims, the term “comprise” and other forms thereof such as the third-person singular form “comprises” and the present participle form “comprising” are construed as an open and inclusive meaning, i.e., “including, but not limited to. In the description of the specification, the terms such as “one embodiment”, “some embodiments”, “exemplary embodiments”, “example” or “some examples” are intended to indicate that specific features, structures, materials or characteristics related to the embodiment(s) or example(s) are included in at least one embodiment or example of the present disclosure. Schematic representations of the above terms do not necessarily refer to the same embodiment(s) or example(s). In addition, the specific features, structures, materials, or characteristics may be included in any one or more embodiments or examples in any suitable manner.

Hereinafter, the terms such as “first” and “second” are used for descriptive purposes only and are not to be construed as indicating or implying the relative importance or implicitly indicating the number of indicated technical features. Thus, features defined with “first” or “second” may explicitly or implicitly include one or more of the features. In the description of the embodiments of the present disclosure, the term “a/the plurality of” means two or more unless otherwise specified.

In the description of some embodiments, the expressions “coupled,” “connected,” and derivatives thereof may be used. For example, the term “connected” may be used in the description of some embodiments to indicate that two or more elements are in direct physical or electrical contact with each other. As another example, the term “coupled” may be used in the description of some embodiments to indicate that two or more elements are in direct physical or electrical contact. However, the term “coupled” or “communicatively coupled” may also mean that two or more elements are not in direct contact with each other, but still cooperate or interact with each other. The embodiments disclosed herein are not necessarily limited to the contents herein.

The phrase “at least one of A, B, and C” has the same meaning as the phrase “at least one of A, B, or C”, both including the following combinations of A, B, and C: only A, only B, only C, a combination of A and B, a combination of A and C, a combination of B and C, and a combination of A, B, and C.

The phrase “A and/or B” includes the following three combinations: only A, only B, and a combination of A and B.

The use of the phrase “applicable to” or “configured to” herein means an open and inclusive expression, which does not exclude devices that are applicable to or configured to perform additional tasks or steps.

It will be understood that when a layer or element is referred to as being on another layer or substrate, the layer or element may be directly on the another layer or substrate, or there may be intermediate layer(s) between the layer or element and the another layer or substrate.

Exemplary embodiments are described herein with reference to sectional views and/or plan views that are schematic illustrations of idealized embodiments. In the accompanying drawings, thicknesses of layers and sizes of regions/areas are enlarged for clarity. Thus, variations in shape with respect to the accompanying drawings due to, for example, manufacturing technologies and/or tolerances may be envisaged. Therefore, the exemplary embodiments should not be construed as being limited to the shapes of the regions shown herein, but including shape deviations due to, for example, manufacturing. For example, an etched region shown to have a rectangular shape generally has a curved feature. Therefore, the regions shown in the accompanying drawings are schematic in nature, and their shapes are not intended to show actual shapes of the regions in a device, and are not intended to limit the scope of the exemplary embodiments.

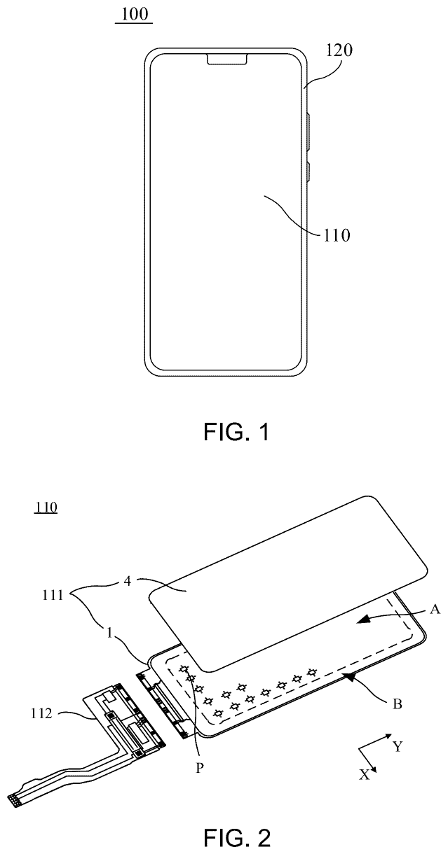

As shown in FIG. 1, some embodiments of the present disclosure provide a display apparatus 100, and the display apparatus 100 may be any apparatus that can display an image whether in motion (e.g., video) or stationary (e.g., a still image), and whether textual or pictorial. More specifically, it is expected that some embodiments of the present disclosure may be implemented in or associated with a variety of electronic apparatuses. The variety of electronic devices may be (but not limited to), for example, a mobile telephone, a wireless device, a personal data assistant (PDA), a hand-held or portable computer, a global positioning system (GPS) receiver/navigator, a camera, an MPEG-4 Part 14 (MP4) video player, a video camera, a game console, a watch, a clock, a calculator, a television monitor, a flat panel display, a computer monitor, a car display (e.g., an odometer display), a navigator, a cockpit controller and/or display, a camera view display (e.g., a rear view camera display in vehicle), an electronic photo, an electronic billboard or signage, a projector, a building structure, and a packaging and aesthetic structure (e.g., a display for an image of a piece of jewelry).

In some embodiments, with continuing reference to FIG. 1, the display apparatus 100 includes a display module 110 and a housing 120.

In some examples, as shown in FIG. 2, the display module 110 includes a display substrate 111, a flexible circuit board 112, and other electronic accessories.

It will be noted that the type of the display substrate 111 has a variety of options, which can be selected and set according to actual needs. As an example, the display substrate 111 may be an electroluminescent display substrate. For example, the display substrate 111 may be an organic light-emitting diode (OLED) display substrate, a quantum dot light-emitting diode (QLED) display substrate, or the like, which is not specifically limited in the embodiments of the present disclosure.

Some embodiments of the present disclosure will be schematically described below by taking an example in which the display substrate 111 is the OLED display substrate.

In some embodiments, as shown in FIG. 2, the display substrate 111 may have a display area A disposed within a dashed box, and a peripheral area B disposed outside the dashed box. Here, the display area A is an area in which the display substrate 111 displays an image, and the peripheral area B is an area in which no image is displayed. The peripheral area B is configured to be provided with display driving circuits, such as a gate driving circuit and a source driving circuit.

It will be noted that the present disclosure does not limit the position of the peripheral area B. For example, the peripheral area BB may be located on one side, two sides or three sides of the display area AA. As another example, the peripheral area BB may be arranged around the display area AA. FIG. 2 exemplarily illustrates the peripheral area B around the display area A as an example.

In some examples, as shown in FIG. 2, the display apparatus 111 includes a plurality of sub-pixels P that are disposed on a side of a substrate 1 and located in the display area A. For example, the plurality of sub-pixels P include at least sub-pixels of a first color, sub-pixels of a second color and sub-pixels of a third color. Here, the first color, the second color, and the third color may be three primary colors (e.g., red, green, and blue).

The plurality of sub-pixels P are arranged in multiple rows and multiple columns in which each row includes multiple sub-pixels P arranged along a first direction X, and each column includes multiple sub-pixels P arranged along a second direction Y. Here, multiple sub-pixels P arranged in one line in the first direction X may be referred to as sub-pixels in a same row, and multiple sub-pixels P arranged in one line in the second direction Y may be referred to as sub-pixels in a same column.

Here, the first direction X intersects the second direction Y. An included angle between the first direction X and the second direction Y may be selected and set according to actual needs. For example, the included angle between the first direction X and the second direction Y may be 85°, 89°, or 90°.

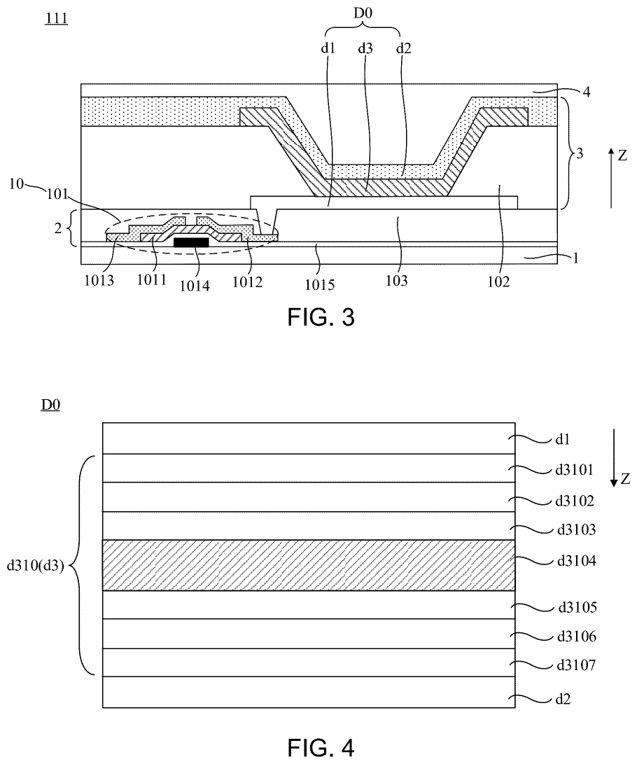

In some embodiments, as shown in FIG. 2 and FIG. 3, the display substrate 111 includes a substrate 1, a circuit structure layer 2, a light-emitting structure layer 3, and an encapsulation layer 4. Here, the circuit structure layer 2 is disposed on the substrate 1, and the circuit structure layer 2 includes a plurality of pixel driving circuits 10, in which the pixel driving circuits 10 each include multiple transistors 101. The light-emitting structure layer 3 is disposed on a side of the circuit structure layer 2 away from the substrate 1, and the light-emitting structure layer 3 includes a plurality of light-emitting devices D0, in which one light-emitting device D0 is correspondingly connected to one pixel driving circuit 10. The encapsulation layer 4 is disposed on a side of the light-emitting structure layer 3 away from the substrate 1, and the encapsulation layer 4 is configured to encapsulate the circuit structure layer 2 and the light-emitting structure layer 3 on the substrate 1.

It will be noted that the types of the transistors 101 included in the pixel driving circuit 10 each have a variety of options. For example, each transistor 101 included in the pixel driving circuit 10 may be a thin film transistor with a bottom gate structure, or may be a thin film transistor with a top gate structure.

In some examples, the multiple transistors 101 included in the pixel driving circuit 10 include one driving transistor, and the driving transistor is electrically connected to the light-emitting device D0.

It will be noted that the driving transistor and the light-emitting device D0 may be directly electrically connected together or indirectly electrically connected together.

In some examples, as shown in FIG. 3, the transistor 101 includes an active layer 1011, a source 1012, a drain 1013, a gate 1014, and a portion of a gate insulating layer 1015. The source 1012 and the drain 1013 are in contact with the active layer 1011. The portion of the gate insulating layer 1015 is disposed between the active layer 1011 and the gate 1014.

On this basis, for example, with continuing reference to FIG. 3, the light-emitting device D0 includes a first electrode d1, a light-emitting functional layer d3 and a second electrode d2 arranged in sequence along a direction Z away from the substrate 1. The first electrode d1 is electrically connected to a source 1012 or drain 1013 of at least one transistor 101 in the multiple transistors 101. FIG. 3 exemplarily illustrates a first electrode d1 electrically connected to a source 1012 of a transistor 101 as an example.

It will be noted that the first electrodes d1 of the multiple light-emitting devices D0 together constitute a first electrode layer, the second electrodes d2 of the multiple light-emitting devices D0 together constitute a second electrode layer, and the light-emitting functional layers d3 of the multiple light-emitting devices D0 together constitute an organic light-emitting layer.

It will be noted that the first electrode layer may be, for example, a block-shaped structure; and the second electrode layer may be, for example, a whole-layer structure and cover the entire display area A; furthermore, the organic light-emitting layer may be, for example, a whole-layer structure, or a block-shaped structure.

The first electrode d1 may be an anode or a cathode, and the second electrode d2 may be a cathode or an anode, accordingly.

In some examples, the first electrode d1 is an anode and the first electrode layer is an anode layer, while the second electrode d2 is a cathode and the second electrode layer is a cathode layer, accordingly. In this case, the light-emitting device D0 is an upright top-emitting light-emitting device. Since the first electrode d1 is not transparent to light, and the second electrode d2 is transparent or semi-transparent, light emitted by the light-emitting functional layer d3 can be emitted from a side of the light-emitting device D0 away from the substrate 1.

It should be understood that the following embodiments are all illustrated exemplarily with the first electrode d1 as the anode and the second electrode d2 as the cathode.

In some examples, the encapsulation layer 4 may be an encapsulation film or an encapsulation substrate.

In some examples, as shown in FIG. 3, the display substrate 111 further includes a pixel defining layer 102, and the pixel defining layer 102 includes a plurality of openings, in which one light-emitting device D0 is disposed in one opening.

In some examples, as shown in FIG. 3, the display substrate 111 further includes a covering layer (CPL) 103 disposed on a side of the first electrode d1 away from the second electrode d2. For example, the material constituting the covering layer 103 may be 4,4′-bis[N-(1-naphthyl)-N-phenylamino]biphenyl (NPB).

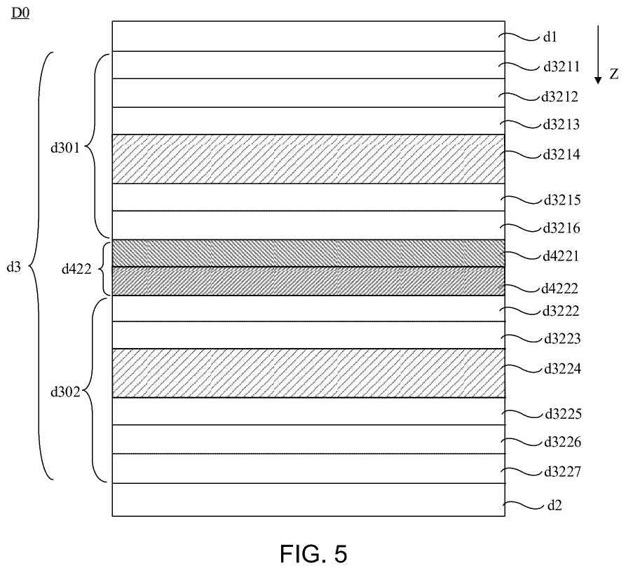

Hereinafter, the structure of the light-emitting device D0 in the display substrate 111 is described in combination with FIG. 4 and FIG. 5.

In some embodiments, the light-emitting functional layer d3 of the light-emitting device D0 includes only one light-emitting unit d310. In this case, as shown in FIG. 4, the light-emitting device D0 includes the first electrode d1, the light-emitting unit d310 and the second electrode d2 that are arranged along the direction Z away from the first electrode d1.

In some examples, referring to FIG. 4, the light-emitting unit d310 includes a hole injection layer (HIL) d3101, a hole transport layer (HTL) d3102, an electron blocking layer (EBL) d3103, a light-emitting layer (EML) d3104, a hole blocking layer (HBL) d3105, an electron transport layer (ETL) d3106, and an electron injection layer (EIL) d3107 along the direction Z away from the first electrode d1.

In this case, in conjunction with FIG. 3 and FIG. 4, the working principle of the above-described display substrate 111 is explained as follows: when the pixel driving circuit 10 operates and transmits a driving voltage to the light-emitting device D0 through a transistor 101 electrically connected to the first electrode d1 thereof, the first electrode d1 can generate positively charged holes due to the action of the electric field, and the second electrode d2 can generate negatively charged electrons due to the action of the electric field. In this case, the holes generated by the first electrode d1 can be injected into the hole transport layer d3102 through the hole injection layer d3101, and enter the light-emitting layer d3104 through the hole transport layer d3102 and the electron blocking layer d3103. Accordingly, the electrons generated by the second electrode d2 can be injected into the electron transport layer d3106 through the electron injection layer d3107, and enter the light-emitting layer d3104 through the electron transport layer d3106 and the hole blocking layer d3105. The holes and electrons in the light-emitting layer d3104 are recombined to form excitons, and the excitons return to the ground state by radiative leaps to emit photons. In this way, the light-emitting device D0 emits light.

In some other embodiments, the light-emitting functional layer d3 of the above-described light-emitting device D0 may include at least two light-emitting units arranged in a stack and at least one stacked connection layer, in which a stacked connection layer is disposed between every two adjacent light-emitting units. In this case, the light-emitting device D0 may be called a stacked light-emitting device.

In this case, every two light-emitting units in the stacked light-emitting device are connected in series through a stacked connection layer, thereby improving the light-emitting efficiency of the stacked light-emitting device and extending the service life of the stacked light-emitting device. Moreover, as the number of the light-emitting units included in the stacked light-emitting device increases, the light-emitting efficiency and service life of the stacked light-emitting device linearly increases.

In some examples, as shown in FIG. 5, the light-emitting functional layer d3 of the above-described light-emitting device D0 includes two light-emitting units arranged in a stack, which are a first light-emitting unit d301 and a second light-emitting unit d302 respectively. In this case, the above-described light-emitting device D0 (i.e., the stacked light-emitting device) includes the first electrode d1, the first light-emitting unit d301, the stacked connection layer d422, the second light-emitting unit d302, and second electrode d2 along the direction Z away from the first electrode d1.

The first light-emitting unit d301 includes a first hole injection layer d3211, a first hole transport layer d3212, a first electron blocking layer d3213, a first light-emitting layer d3214, a first hole blocking layer d3215, and a first electron transport layer d3216 that are arranged along the direction Z away from the first electrode d1. The second light-emitting unit d302 includes a second hole transport layer d3222, a second electron blocking layer d3223, a second light-emitting layer d3224, a second hole blocking layer d3225, a second electron transport layer d3226, and a second electron injection layer d3227 along the direction Z away from the first electrode d1.

The stacked connection layer d422 includes an N-type charge generation layer d4221 and a P-type charge generation layer d4222 arranged in a stack. The N-type charge generation layer d4221 is disposed on a side of the stacked connection layer d422 proximate to the first electrode d1; and the P-type charge generation layer d4222 is disposed on a side of the stacked connection layer d422 proximate to the second electrode d2.

In this case, in conjunction with FIG. 3 and FIG. 5, the working principle of the above-described display substrate 111 is explained as follows: when the pixel driving circuit 10 operates, the driving voltage may be transmitted to the stacked light-emitting device through the transistor 101 electrically connected to the first electrode d1. In this case, due to the action of the electric field, the first electrode d1 can generate holes, the second electrode d2 can generate electrons, and a contact region between the N-type charge generation layer d4221 and the P-type charge generation layer d4222 can generate holes and electrons. In this case, the holes generated by the first electrode d1 can enter the first light-emitting layer d3214 through the first hole injection layer d3211, the first hole transport layer d3212, and the first electron blocking layer d3213 in sequence. Accordingly, the electrons generated in the contact region between the N-type charge generation layer d4221 and the P-type charge generation layer d4222 may enter into the first light-emitting layer d3214 through the N-type charge generation layer d4221, the first electron transport layer d3216, and the first hole blocking layer d3215 in sequence. Similarly, the holes generated in the contact region between the N-type charge generation layer d4221 and the P-type charge generation layer d4222 may enter into the second light-emitting layer d3224 through the P-type charge generation layer d4222, the second hole transport layer d3222, and the second electron blocking layer d3223 in sequence. The electrons generated by the second electrode d2 may enter the second light-emitting layer d3224 through the second electron injection layer d3227, the second electron transport layer d3226, and the second hole blocking layer d3225 in sequence. In this way, the holes and electrons in the first light-emitting layer d3214, as well as the holes and electrons in the second light-emitting layer d3224 are recombined to form excitons, so the stacked light-emitting device emits light.

It will be noted that the N-type charge generation layer d4221 has electron injection capability, and the P-type charge generation layer d4222 has hole injection capability. Therefore, the N-type charge generation layer d4221 can be used as the electron injection layer in the first light-emitting unit d301, and the P-type charge generation layer d4222 can be used as the hole injection layer in the second light-emitting unit d302. That is, in the above-described stacked light-emitting device, the first light-emitting unit d301 may not have an additional electron injection layer, and the second light-emitting unit d302 may not have an additional hole injection layer.

Of course, in the above-described stacked light-emitting device, the first light-emitting unit d301 may be additionally provided therein with an electron injection layer located between the N-type charge generation layer d4221 and the first electron transport layer d3216, and similarly, the second light-emitting unit d302 may also be additionally provided therein with a hole injection layer located between the P-type charge generation layer d4222 and the second hole transport layer d3222, which are not limited by the present disclosure.

However, the inventors of the present disclosure have found upon study that: since physical parameters of both the N-type charge generation layer d4221 and the P-type charge generation layer d4222 in the above-described stacked light-emitting device in the related art have not been reasonably designed, the two cannot be effectively cooperated with each other. Thus, the holes and electrons generated in the contact region between the N-type charge generation layer d4221 and the P-type charge generation layer d4222 in the stacked connection layer d422 may be quenched, making it difficult to ensure that the number of the electrons and holes in the stacked connection layer d422 supplied to two light-emitting units adjacent thereto (e.g., the above-described first light-emitting unit d301 and second light-emitting unit d302), ultimately resulting in the reduced light-emitting efficiency and service life of the stacked light-emitting device.

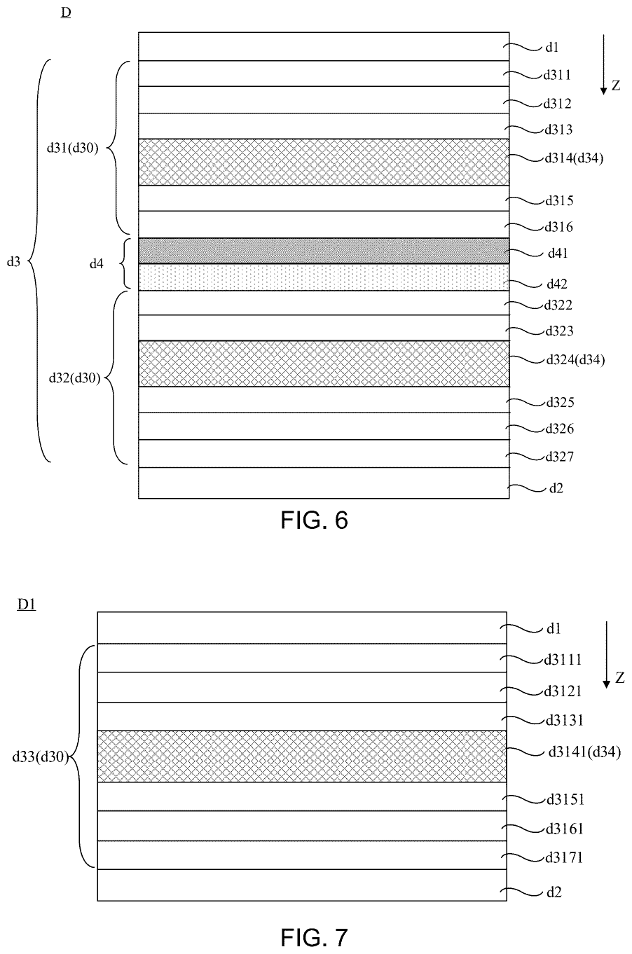

In light of this, as shown in FIG. 6, embodiments of the present disclosure provide a stacked light-emitting device D. The stacked light-emitting device D includes a first electrode d1, a second electrode d2, at least two light-emitting units d30, and at least one stacked connection layer d4. The at least two light-emitting units d30 are stacked between the first electrode d1 and the second electrode d2. A stacked connection layer d4 is disposed between every two adjacent light-emitting units d30, and the stacked connection layer d4 includes an N-type charge generation layer d41 and a P-type charge generation layer d42 disposed in a stack. Here, the N-type charge generation layer d41 is of a doped binary structure including a first host material and a first guest material; the P-type charge generation layer d42 is of a doped binary structure including a second host material and a second guest material; an absolute value of a difference between a highest occupied molecular orbital (HOMO) energy level of the second host material and a highest occupied molecular orbital (HOMO) energy level of the first host material is greater than 0.3 electron volts; and an absolute value of a difference between a lowest unoccupied molecular orbital (LUMO) energy level of the second host material and a lowest unoccupied molecular orbital (LUMO) energy level of the first host material is greater than 0.1 electron volts.

To sum up, in the stacked light-emitting device D provided by the embodiments of the present disclosure, the absolute value of the difference between the HOMO energy level of the second host material and the HOMO energy level of the first host material is limited to be greater than 0.3 electron volts, and the absolute value of the difference between the LUMO energy level of the second host material and the LUMO energy level of the first host material is limited to be greater than 0.1 electron volts, which may prevent the electrons and holes generated in the contact region between the N-type charge generation layer d41 and the P-type charge generation layer d42 from being transferred in the reverse direction (i.e., the electrons generated in the contact region are prevented from being transferred toward the P-type charge generation layer d42 and the holes generated in the contact region are prevented from being transferred toward the N-type charge generation layer d41), thereby preventing the electrons and holes generated in the contact region from being quenched due to the reverse direction of transmission which in turn ensures that each stacked connection layer d4 provides a stable number of carriers to the two light-emitting units adjacent thereto, ultimately enhancing the light-emitting efficiency of the stacked light-emitting device D.

It will be noted that the absolute value of the difference between the HOMO energy level of the second host material and the HOMO energy level of the first host material is greater than 0.3 electron volts, which may mean that the difference between the HOMO energy level of the second host material and the HOMO energy level of the first host material is greater than 0.3 electron volts, and in this case, the HOMO energy level of the second host material is greater than the HOMO energy level of the first host material; alternatively, which may mean that the difference between the HOMO energy level of the second host material and the HOMO energy level of the first host material is less than minus 0.3 electron volts, and in this case, the HOMO energy level of the second host material is less than the HOMO energy level of the first host material.

Similarly, the absolute value of the difference between the LUMO energy level of the second host material and the LUMO energy level of the first host material is greater than 0.1 electron volts, which may mean that the difference between the LUMO energy level of the second host material and the LUMO energy level of the first host material is greater than 0.1 electron volts, and in this case, the LUMO energy level of the second host material is greater than the LUMO energy level of the first host material; alternatively, which may mean that the difference between the LUMO energy level of the second host material and the LUMO energy level of the first host material is less than minus 0.1 electron volts, and in this case, the LUMO energy level of the second host material is less than the LUMO energy level of the first host material.

It will be noted that in the stacked light-emitting device D provided by the embodiments of the present disclosure, the number of the light-emitting units d30 can be selected as needed, and the embodiments of the present disclosure do not limit this. For example, the number of the light-emitting units d30 is two, in which case the number of the stacked connection layer d4 is one. In this way, the manufacturing cost of the stacked light-emitting device D may be saved. As another example, the number of the light-emitting units d30 is three, in which case the number of the stacked connection layers d4 is two. In this way, the light-emitting efficiency of the stacked light-emitting device D may be improved and the service life of the stacked light-emitting device D may be extended.

In some examples, referring to FIG. 6, the stacked light-emitting device D includes a first electrode d1, a first light-emitting unit d31, a stacked connection layer d4, a second light-emitting unit d32, and a second electrode d2 that are arranged along the direction Z away from the first electrode d1.

The first light-emitting unit d31 includes a first hole injection layer d311, a first hole transport layer d312, a first electron blocking layer d313, a first light-emitting layer d314, a first hole blocking layer d315, and a first electron transport layer d316 that are arranged along the direction Z away from the first electrode d1. The second light-emitting unit d32 includes a second hole transport layer d322, a second electron blocking layer d323, a second light-emitting layer d324, a second hole blocking layer d325, a second electron transport layer d326, and a second electron injection layer d327 that are arranged along the direction Z away from the first electrode d1.

Here, the N-type charge generation layer d41 is disposed on a side of the stacked connection layer d4 proximate to the first electrode d1, and the P-type charge generation layer d42 is disposed on a side of the stacked connection layer d4 proximate to the second electrode d2.

In the above embodiments, the N-type charge generation layer d41 serves to inject the electrons generated in the contact region between the N-type charge generation layer d41 and the P-type charge generation layer d42 into the first electron transport layer d316, while the P-type charge generation layer d42 serves to inject the holes generated in this contact region into the second hole transport layer d322. Therefore, the absolute value of the difference between the HOMO energy level of the second host material and the HOMO energy level of the first host material is limited to be greater than 0.3 electron volts, and the absolute value of the difference between the LUMO energy level of the second host material and the LUMO energy level of the first host material is limited to be greater than 0.1 electron volts, which may prevent the electrons and holes generated in the contact region between the N-type charge generation layer d41 and the P-type charge generation layer d42 from being transferred in the reverse direction (i.e., the electrons generated in the contact region are prevented from being transferred toward the P-type charge generation layer d42 and the holes generated in the contact region are prevented from being transferred toward the N-type charge generation layer d41), thereby preventing the electrons and holes generated in the contact region from being quenched due to the reverse direction of transmission which in turn ensures that each stacked connection layer d4 provides a stable number of carriers to the two light-emitting units adjacent thereto, ultimately enhancing the light-emitting efficiency of the stacked light-emitting device D.

It will be noted that the embodiments of the present disclosure do not limit a material of either the first electrode d1 or the second electrode d2.

In some examples, the material of the first electrode d1 is a metal. For example, the material of the first electrode d1 may be selected from at least one of: silver (Ag), magnesium (Mg), copper (Cu), aluminum (AI), platinum (Pt), palladium (Pd), gold (Au), nickel (Ni), neodymium (Nd), iridium (Ir), chromium (Cr), lithium (Li), calcium (Ca), Ca—LiF alloy, Al—LiF alloy, molybdenum (Mo), titanium (Ti), indium (In), tin (Sn), and zinc (Zn).

In some examples, the material of the second electrode d2 is a metal or an inorganic material. For example, in a case where the material of the second electrode d2 is the metal, the material of the second electrode d2 may be silver (Ag), magnesium (Mg), ytterbium (Yb), lithium (Li), or calcium (Ca); and in a case where the material of the second electrode d2 is the inorganic material, the material of the second electrode d2 may be lithium oxide (Li₂O), calcium oxide (CaO), lithium fluoride (LiF), or magnesium fluoride (MgF₂).

In some embodiments, the first guest material of the N-type charge generation layer d41 includes at least one of a metal or an organic matter.

In some examples, the first guest material of the N-type charge generation layer d41 is the metal, and in this case, an absolute value of a difference between a work function of the first guest material and the LUMO energy level of the first host material is less than 1.0 electron volts.

In the above embodiments, the first guest material serves to give electrons on the first guest material to the first host material, to enable the electrons to be transported in the N-type charge generation layer d41 through the first host material, and to be injected into the first electron transport layer d316 adjacent to the N-type charge generation layer d41. Moreover, the closer the work function of the first guest material is to the LUMO energy level of the first host material, the less energy is required for transferring electrons between the first guest material and the first host material, and the easier it is for the electrons on the first guest material to be transferred to the first host material. In this way, the number of the electrons injected into the first electron transport layer d316 by the N-type charge generation layer d41 will increase, and thus, the probability of generating excitons by electron-hole recombination in the first light-emitting layer d314 will increase. Thus, in the stacked light-emitting device D provided in the above embodiments, the probability of generating the excitons by the electron-hole recombination in the first light-emitting layer d314 is increased by limiting a differential relationship between the work function of the first guest material and the LUMO energy level of the first host material, which improves the light-emitting efficiency of the stacked light-emitting device D.

It will be noted that the absolute value of the difference between the work function of the first guest material and the LUMO energy level of the first host material is less than 1.0 electron volts, which may mean that: the difference between the work function of the first guest material and the LUMO energy level of the first host material is greater than or equal to zero and less than 1.0 electron volts, and in this case, the work function of the first guest material is greater than or equal to the LUMO energy level of the first host material; alternatively, which may mean that the difference between the work function of the first guest material and the LUMO energy level of the first host material is greater than minus 1.0 electron volts and less than or equal to zero, and in this case, the work function of the first guest material is less than or equal to the LUMO energy level of the first host material.

In some other examples, the first guest material of the N-type charge generation layer d41 is the organic matter, and in this case, an absolute value of a difference between the HOMO energy level of the first guest material and the LUMO energy level of the first host material is less than 1.0 electron volts.

In the above embodiments, the first guest material serves to give electrons on the first guest material to the first host material, to enable the electrons to be transported in the N-type charge generation layer d41 through the first host material, and to be injected into the first electron transport layer d316 adjacent to the N-type charge generation layer d41. Moreover, the closer the HOMO energy level of the first guest material is to the LUMO energy level of the first host material, the less energy is required for transferring electrons between the first guest material and the first host material, and the easier it is for the electrons on the first guest material to be transferred to the first host material. In this way, the number of the electrons injected into the first electron transport layer d316 by the N-type charge generation layer d41 will increase, and thus, the probability of generating excitons by electron-hole recombination in the first light-emitting layer d314 will increase. Thus, in the stacked light-emitting device D provided in the above embodiments, the probability of generating the excitons by the electron-hole recombination in the first light-emitting layer d314 is increased by limiting a differential relationship between the HOMO energy level of the first guest material and the LUMO energy level of the first host material, which improves the light-emitting efficiency of the stacked light-emitting device D.

It will be noted that the absolute value of the difference between the HOMO energy level of the first guest material and the LUMO energy level of the first host material is less than 1.0 electron volts, which may mean that: the difference between the HOMO energy level of the first guest material and the LUMO energy level of the first host material is greater than or equal to zero and less than 1.0 electron volts, and in this case, the HOMO energy level of the first guest material is greater than or equal to the LUMO energy level of the first host material; alternatively, which may mean that the difference between the HOMO energy level of the first guest material and the LUMO energy level of the first host material is greater than minus 1.0 electron volts and less than or equal to zero, and in this case, the HOMO energy level of the first guest material is less than or equal to the LUMO energy level of the first host material.

It will be noted that the embodiments of the present disclosure do not limit the method of obtaining the doped binary structure of the N-type charge generation layer d41. For example, the first guest material may be doped into the first host material by an ion implantation method or a diffusion method to obtain the doped binary structure.

In some embodiments, an absolute value of a difference between the LUMO energy level of the second guest material and the HOMO energy level of the second host material is less than 0.5 electron volts.

In the above embodiments, the second guest material serves to give holes on the second guest material to the second host material, to enable the holes to be transported in the P-type charge generation layer d42 through the second host material, and to be injected into the second hole transport layer d322 adjacent to the P-type charge generation layer d42. Moreover, the closer the LUMO energy level of the second guest material is to the HOMO energy level of the second host material, the less energy is required for transferring holes between the second guest material and the second host material, and the easier it is for the holes on the second guest material to be transferred to the second host material. In this way, the number of the holes injected into the second holes transport layer d322 by the P-type charge generation layer d42 will increase, and thus, the probability of generating excitons by electron-hole recombination in the second light-emitting layer d324 will increase. Thus, in the stacked light-emitting device D provided in the above embodiments, the probability of generating the excitons by the electron-hole recombination in the second light-emitting layer d324 is increased by limiting a differential relationship between the LUMO energy level of the second guest material and the HOMO energy level of the second host material, which improves the light-emitting efficiency of the stacked light-emitting device D.

It will be noted that the absolute value of the difference between the LUMO energy level of the second guest material and the HOMO energy level of the second host material is less than 0.5 electron volts, which may mean that the difference between the LUMO energy level of the second guest material and the HOMO energy level of the second host material is greater than or equal to zero and less than 0.5 electron volts, and in this case, the LUMO energy level of the second guest material is greater than or equal to the HOMO energy level of the second host material; alternatively, which may mean that the difference between the LUMO energy level of the second guest material and the HOMO energy level of the second host material is greater than minus 0.5 electron volts and less than or equal to zero, and in this case, the LUMO energy level of the second guest material is greater than or equal to the HOMO energy level of the second host material.

It will be noted that the embodiments of the present disclosure do not limit the method of obtaining the doped binary structure of the P-type charge generation layer d42. For example, the second guest material may be doped into the second host material by an ion implantation method or a diffusion method to obtain the doped binary structure.

In the above-described embodiments, limitations between physical parameters (e.g., limitations on the absolute value of the difference between the HOMO energy level of the second host material and the HOMO energy level of the first host material) of the materials constituting all structures of the stacked connection layer d4 are mainly described. Hereinafter, an exemplary description of each material that satisfies the above limitations between the physical parameters is provided.

In some embodiments, a structure of the first host material of the N-type charge generation layer d41 has a conjugated fragment; and the conjugated fragment has at least two benzene rings, and all of the benzene rings in the conjugated fragment are of a π-π conjugated structure.

In these embodiments, the structure of the first host material of the N-type charge generation layer d41 has a conjugated fragment. On this basis, since the electron cloud overlapping portion between molecules of the conjugated structure is large, it is conducive to the hopping-electron transport between molecules. Therefore, in the stacked light-emitting device D provided in the embodiments of the present disclosure, by adopting a material having a conjugated fragment in its structure as the host material of the N-type charge generation layer d41, it is possible to enhance the smoothness of transporting the electrons in the N-type charge generation layer d41, i.e., to enhance the electron mobility of the N-type charge generation layer d41, thereby enhancing the efficiency of transporting the electrons through the N-type charge generation layer d41 to the electron transport layer d316, and thereby enhancing the light-emitting efficiency of the stacked light-emitting device D.

It will be noted that the present disclosure does not limit the number of the benzene rings contained in the above-described conjugated fragment, as long as all of the benzene rings contained therein are π-π conjugated structures.

In some examples, the above-described conjugated fragment has two benzene rings, and the two benzene rings are of a π-π conjugated structure. In this case, the conjugated fragment is a naphthalene fragment, its chemical formula is C₁₀H₈, and its structural formula is:

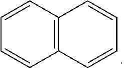

In some other examples, the above-described conjugated fragment has three benzene rings, and the three benzene rings are of a π-π conjugated structure. In this case, the conjugated fragment is an anthracene fragment, its chemical formula is C₁₄H₁₀, and its structural formula is:

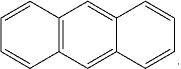 [SMILES: c1ccc2cc3ccccc3cc2c1]

In yet some other examples, the above-described conjugated fragment has five benzene rings, and the five benzene rings are of a π-π conjugated structure. In this case, the conjugated fragment is a pentacene fragment, its chemical formula is C₂₂H₁₄, and its structural formula is:

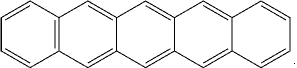 [SMILES: c1ccc2cc3cc4cc5ccccc5cc4cc3cc2c1]

It will be noted that the structure of the first host material of the N-type charge generation layer d41 further has at least one substituent connected to the conjugated fragment.

In some examples, the at least one substituent is each independently selected from: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₃₀ aryloxy group.

In some embodiments, at least one substituent on the conjugated fragment has a phosphorus oxygen group (P═O group).

In these embodiments, the structure of the first host material of the N-type charge generation layer d41 has the phosphorus oxygen group (P═O group). On this basis, since the phosphorus oxygen group is a strong electron-withdrawing group (a strong electron-withdrawing group is a substituent group that externally exhibits a positive electric field, which tends to withdraw electrons). Therefore, in the stacked light-emitting device D provided by the embodiments of the present disclosure, by adopting the material with the phosphorus oxygen group in its structure as the first host material, it is possible to enhance the electron injection capability of the N-type charge generation layer d41, thereby further improving the electron mobility of the N-type charge generation layer d41, and thereby improving the light-emitting efficiency of the stacked light-emitting device D.

In some embodiments, the first host material of the N-type charge generation layer d41 has a structure shown in the following Formula (I):

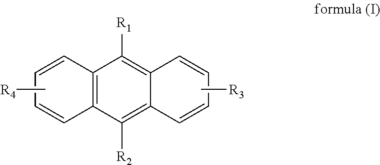

where R₁, R₂, R₃ and R₄ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, a substituted or unsubstituted C₆ to C₃₀ aryloxy group, and a structure shown in the following Formula (II):
where at least one of R₁, R₂, R₃ and R₄ has the structure shown in the following Formula (II):

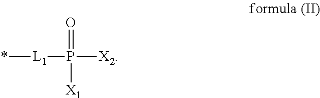

where * indicates a site connected to a carbon atom.
L₁ is selected from any one of: single bond, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C to Co aralkyl group, a substituted or unsubstituted C₁ to C₂₀ alkoxy group, and a substituted or unsubstituted C₆ to C₆₀ aryloxy group.
X₁ are X₂ each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted Cs to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₃₀ aryloxy group.

In these embodiments, the first host material of the N-type charge generation layer d41 has the structure as shown in the above Formula (I), that is, the structure of the first host material has the anthracene fragment (with the molecular formula of C₁₄H₁₀, and the structural formula of

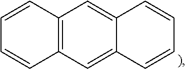 [SMILES: c1ccc2cc3ccccc3cc2c1]

which is of a large conjugated rigid structure. On this basis, since the electron cloud overlapping portion between molecules of the conjugated structure is large, it is conducive to the hopping-electron transport between molecules. Therefore, in the stacked light-emitting device D provided in the embodiments of the present disclosure, by adopting a material having the anthracene fragment in its structure as the host material of the N-type charge generation layer d41, it is possible to enhance the smoothness of transporting the electrons in the N-type charge generation layer d41, i.e., to enhance the electron mobility of the N-type charge generation layer d41, thereby enhancing the efficiency of transporting the electrons through the N-type charge generation layer d41 to the electron transport layer d316, and thereby enhancing the light-emitting efficiency of the stacked light-emitting device D. In addition, at least one of the substituent groups R₁, R₂, R₃ and R₄ on the first host material of the N-type charge generation layer d41 has the structure of Formula (II), that is, the structure of the first host material has a phosphorus oxygen group (P═O group). On this basis, since the phosphorus oxygen group is a strong electron-withdrawing group, in the stacked light-emitting device D provided by the embodiments of the present disclosure, by adopting the material having the phosphorus oxygen group in its structure as the first host material, it is possible to enhance the electron injection capability of the N-type charge generation layer d41, thereby further enhancing the electron mobility of the N-type charge generation layer d41, and thereby enhancing the light-emitting efficiency of the stacked light-emitting device D.

It will be noted that the embodiments of the present disclosure do not limit the substitution sites of the above-described R₃ and R₄.

For example, the first host material has a structure shown in the following Formula (IV):

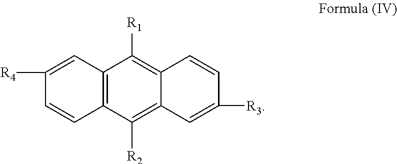

Alternatively, the first host material has a structure shown in the following Formula (V):

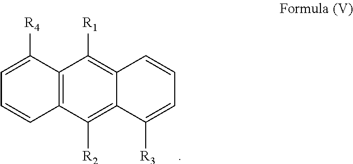

In some embodiments, at least one of R₃ and R₄ has the structure shown in the above Formula (II).

In the stacked light-emitting device D provided in the above embodiments, by using a material having a tail group (R₃ and/or R₄) with the structure as shown in Formula (II) (the structure of the tail group has the phosphorus oxygen group) as the first host material, it is possible to further enhance the electron injection capability of the N-type charge generation layer d41, and thereby further enhance the electron mobility of the N-type charge generation layer d41, which in turn enhances the light-emitting efficiency of the stacked light-emitting device D.

In some embodiments, both R₃ and R₄ have the structure shown in the above Formula (II).

In the stacked light-emitting device D provided in the above embodiments, by using a material having tail groups (R₃ and R₄) both with the structure as shown in Formula (II) (the structure of the tail group has the phosphorus oxygen group) as the first host material, it is possible to even further enhance the electron injection capability of the N-type charge generation layer d41, and thereby even further enhance the electron mobility of the N-type charge generation layer d41, which in turn enhances the light-emitting efficiency of the stacked light-emitting device D.

In some embodiments, the first host material has a structure shown in any one of the following Formula (1-1) to Formula (1-10):

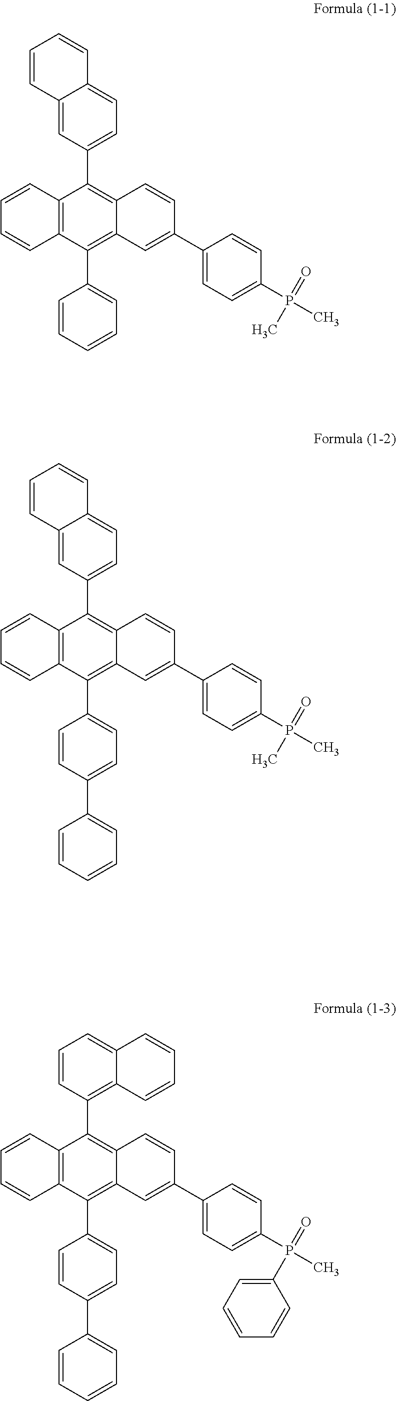 [SMILES: CP(C)(=O)c1ccc(-c2ccc3c(-c4ccc5ccccc5c4)c4ccccc4c(-c4ccccc4)c3c2)cc1 ; CP(C)(=O)c1ccc(-c2ccc3c(-c4ccc5ccccc5c4)c4ccccc4c(-c4ccc(-c5ccccc5)cc4)c3c2)cc1 ; CP(=O)(c1ccccc1)c1ccc(-c2ccc3c(-c4cccc5ccccc45)c4ccccc4c(-c4ccc(-c5ccccc5)cc4)c3c2)cc1]

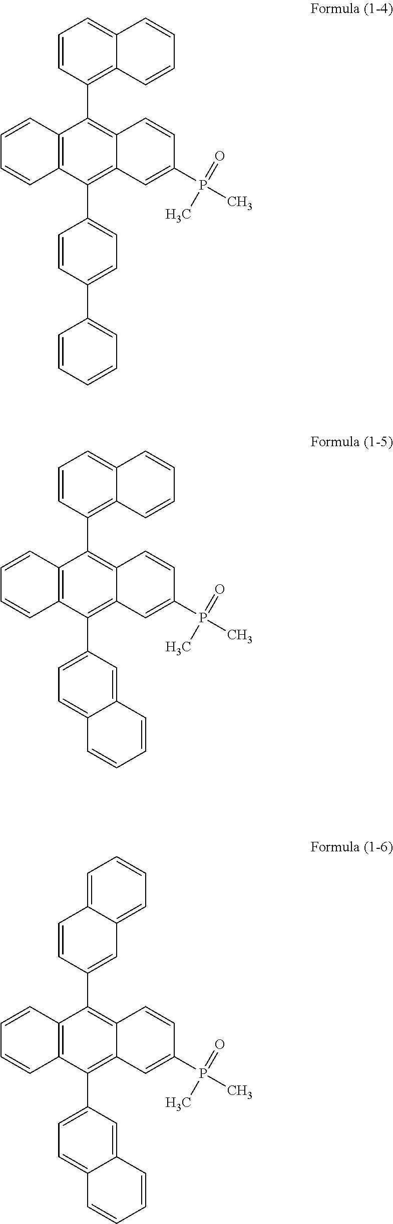 [SMILES: CP(C)(=O)c1ccc2c(-c3cccc4ccccc34)c3ccccc3c(-c3ccc(-c4ccccc4)cc3)c2c1 ; CP(C)(=O)c1ccc2c(-c3cccc4ccccc34)c3ccccc3c(-c3ccc4ccccc4c3)c2c1 ; CP(C)(=O)c1ccc2c(-c3ccc4ccccc4c3)c3ccccc3c(-c3ccc4ccccc4c3)c2c1]

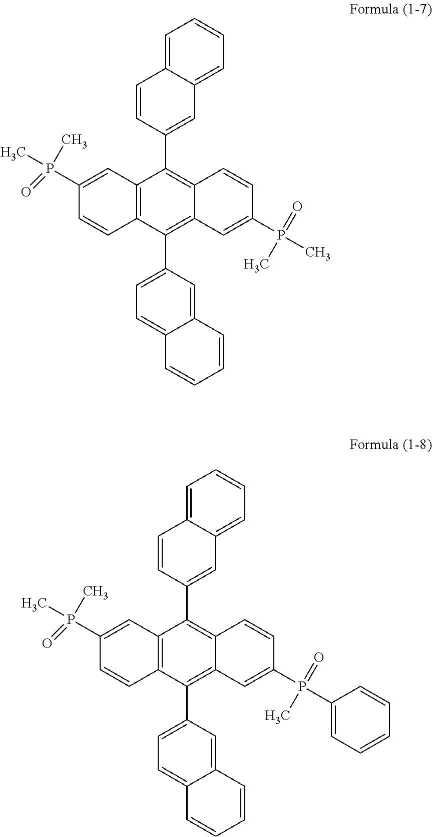

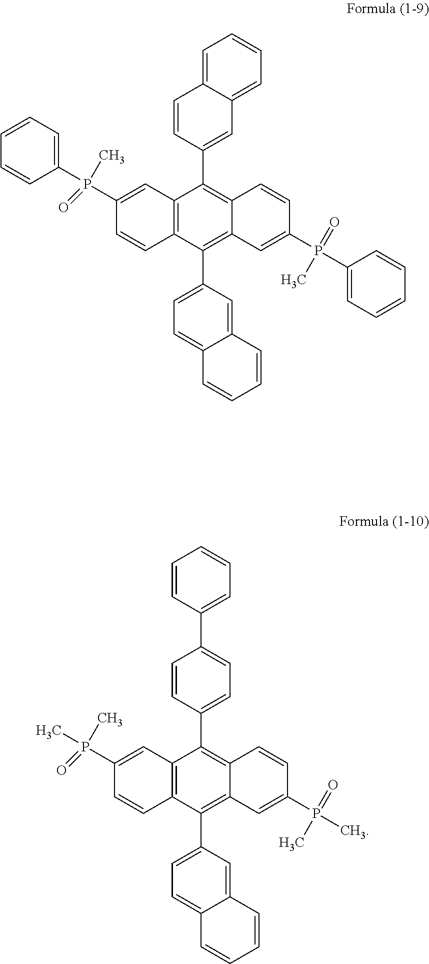 [SMILES: CP(=O)(c1ccccc1)c1ccc2c(-c3ccc4ccccc4c3)c3cc(P(C)(=O)c4ccccc4)ccc3c(-c3ccc4ccccc4c3)c2c1 ; CP(C)(=O)c1ccc2c(-c3ccc4ccccc4c3)c3cc(P(C)(C)=O)ccc3c(-c3ccc(-c4ccccc4)cc3)c2c1]

It will be noted that the materials shown in Formula (1-1) to Formula (1-10) above are only used as examples of the first host material, and any material satisfying Formula (1) above can be used as the first host material in the embodiments of the present disclosure.

In the above embodiments, in a case where the first host material has the structure shown in the above Formula (1-1) to Formula (1-10), the phosphorus oxygen group present in the structure of the tail group (R₃ and/or R₄) of the first host material may further enhance the electron injection capability of the N-type charge generation layer d41, thereby further enhancing the electron mobility of the N-type charge generation layer d41, and thereby enhancing the light-emitting efficiency of the stacked light-emitting device D.

In some examples, a method of preparing the material shown in the above Formula (1-1) may include steps S11 and S12.

In S11, argon gas is introduced into a flask with a capacity of 500 mL; 3.06 g of raw material 1 with a structure as shown in Formula A and 3.23 g of raw material 2 with a structure as shown in Formula B are added into the flask; and 0.236 g of tetrakis(triphenylphosphine)palladium, 20 mL of 1,2-dimethoxyethane, 20 mL of toluene, and 20 mL of aqueous sodium carbonate 2M (i.e., 20 mL of aqueous sodium carbonate with a volume molar concentration of 2 mol/L) are added into the flask; the substances within the flask are heated to 150° C. with reflux reaction for 10 hours; the product obtained after the reflux reaction is cooled to room temperature, and then the product is filtered, cooled and precipitated as a solid; and the solid obtained by filtration is washed with water and methanol, and then the solid is recrystallized with toluene to obtain intermediate 1 with a structure shown in Formula C.

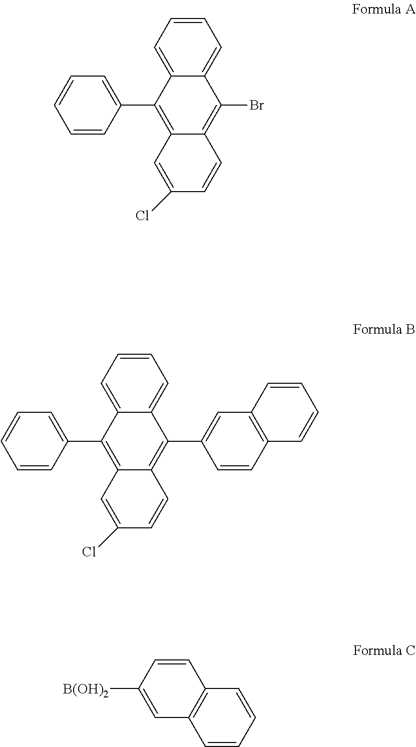 [SMILES: Clc1ccc2c(Br)c3ccccc3c(-c3ccccc3)c2c1 ; Clc1ccc2c(-c3ccc4ccccc4c3)c3ccccc3c(-c3ccccc3)c2c1 ; OB(O)c1ccc2ccccc2c1]

Here, the reaction equation in the above S11 is as follows:

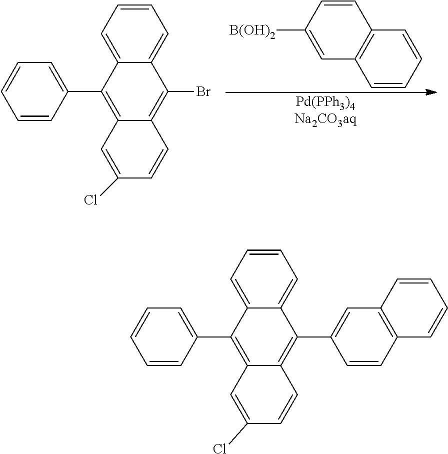 [SMILES: Clc1ccc2c(Br)c3ccccc3c(-c3ccccc3)c2c1 ; OB(O)c1ccc2ccccc2c1 ; "Pd(PPh3)4Na2CO3aq" ; Clc1ccc2c(-c3ccc4ccccc4c3)c3ccccc3c(-c3ccccc3)c2c1]

In S12, nitrogen gas is introduced into a three-necked flask with a capacity of 500 mL; 0.02 mol of intermediate 1, 0.02 mol of raw material 3 with a structure as shown in Formula D, 0.002 mol of dimethylformamide (DMF), and 0.002 mol of palladium acetate (Pd(OAc)₂) are added into the three-necked flask, and the substances are stirred; then 0.01 mol of aqueous K₃PO₄ is added into the flask, and the substances within the flask are heated to 150° C., with reflux reaction for 24 hours, and samples are taken for thin-layer chromatography until the reaction is determined to be complete; and after the product obtained from the above reflux reaction is cooled to room temperature, the cooled product is extracted with dichloromethane and the extract liquor obtained is dried with anhydrous sodium sulfate, the solid precipitated after drying is filtered off, and then the filtrate obtained is subjected to rotary evaporation, and finally the evaporated substance is purified using a silica gel column, to obtain a phosphorus oxygen derivative shown in the above Formula (1-1).

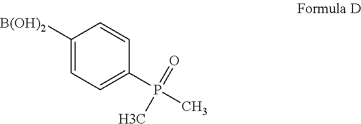

Here, the reaction equation in the above S12 is as follows:

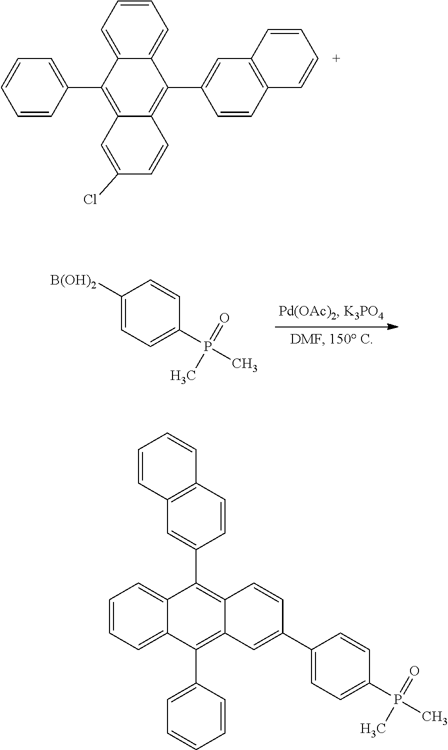 [SMILES: Clc1ccc2c(-c3ccc4ccccc4c3)c3ccccc3c(-c3ccccc3)c2c1 ; CP(C)(=O)c1ccc(B(O)O)cc1 ; CP(C)(=O)c1ccc(-c2ccc3c(-c4ccc5ccccc5c4)c4ccccc4c(-c4ccccc4)c3c2)cc1]

It will be noted that since the materials shown in the above Formula (1-1) to Formula (1-10) are phosphorus oxygen derivatives having the same general formula, the method of preparing the materials shown in the above Formula (1-2) to Formula (1-10) is similar to that of the material shown in the Formula (1-1), which will not be described herein.

In some examples, the first guest material described above may be an organic substance having a strong electron-donating group, a metal (e.g., an alkali metal), or a metal-containing compound. Here, the strong electron-donating group is a substituent group that externally exhibits a negative electric field, which tends to donate electrons.

For example, the first guest material may be selected from leuco crystal violet (LCV), lithium (Li), sodium (Na), potassium (K), rubidium (Rb), cesium (Cs), francium (Fr), magnesium (Mg), calcium (Ca), ytterbium (Yb), or lithium fluoride (LiF).

In a case where the N-type charge generation layer d41 in the stacked light-emitting device D is composed of the first host material and the first guest material provided in the embodiments described above, it is possible to satisfy a physical parameter requirement of “the absolute value of the difference between the work function of the first guest material and the LUMO energy level of the first host material being less than 1.0 electron volts” or satisfy a physical parameter requirement of “the absolute value of the difference between the HOMO energy level of the first guest material and the LUMO energy level of the first host material being less than 1.0 electron volts”, so that the number of electrons injected by the N-type charge generation layer d41 into the first electron transport layer d316 may be increased, thereby increasing the probability of generating excitons by electron-hole recombination in the first light-emitting layer d314, ultimately increasing the light-emitting efficiency of the stacked light-emitting device D.

In some examples, the P-type charge generation layer d42 is of a doped binary structure including a second host material and a second guest material.

For example, the second host material may be an aromatic amine-like material, a dimethyl fluorene material, or a carbazole-like material that has hole transport properties. For example, the second host material may be selected from 4,4′-bis[N-(1-naphthyl)-N-phenylamino]biphenyl (NPB), N,N′-bis(3-methylphenyl)-N,N′-diphenyl-[1,1′-biphenyl]-4,4′-diamine (TPD), 4-phenyl-4′-(9-phenylfluoren-9-yl)triphenylamine (BAFLP), 4,4′-bis[N-(9,9-dimethylfluoren-2-yl)-N-phenylamino]biphenyl (DFLDPBi), 4,4′-bis(9-carbazolyl)biphenyl (CBP), 9-phenyl-3-[4-(10-phenyl-9-anthryl)phenyl]-9H-carbazole (PCzPA), or 4,4′,4″-tris(N-3-methylphenyl-N-phenylamino)triphenylamine (m MTDATA).

For example, the second guest material described above may be an organic material having a strong electron-withdrawing group. For example, the second guest material may be selected from hexacyano hexaazatriphenylene, 2,3,5,6-tetrafluoro-7,7,8,8-tetracyano-p-quinodimethane (F4TCNQ), or 1,2,3-tris[(cyano)(4-cyano-2,3, 5,6-tetrafluorophenyl)methylene]cyclopropane.

In a case where in the stacked light-emitting device D, the N-type charge generation layer d41 is composed of the first host material and the first guest material provided in the embodiments described above, and the P-type charge generation layer d42 is composed of the second host material and the second guest material provided in the embodiments described above, it is possible to satisfy a physical parameter requirement of “the absolute value of the difference between the HOMO energy level of the second host material and the HOMO energy level of the first host material being greater than 0.3 electron volts, and the absolute value of the difference between the LUMO energy level of the second host material and the LUMO energy level of the first host material being greater than 0.1 electron volts”, which may prevent the electrons and holes generated in the contact region between the N-type charge generation layer d41 and the P-type charge generation layer d42 from being transferred in the reverse direction, thereby preventing the electrons and holes generated in the contact region from being quenched due to the reverse direction of transmission which in turn ensures that each stacked connection layer d4 provides a stable number of carriers to the two light-emitting units d30 adjacent thereto, ultimately enhancing the light-emitting efficiency of the stacked light-emitting device D.

In some embodiments, the light-emitting layer d34 in the light-emitting unit d30 is of a doped binary structure including a third host material and a third guest material. The structure of the third host material has a conjugated fragment; the conjugated fragment has at least two benzene rings, and all of the benzene rings in the conjugated fragment are of a π-π conjugated structure.

In these embodiments, in the stacked light-emitting device D, a material having the above-described conjugated fragment in its structure is selected as the host material of the light-emitting layer d34 (i.e., the third host material). On this basis, since the conjugated fragment has at least two benzene rings (aromatic rings), which is characterized by high fluorescence quantum yields, adopting the material having the conjugated fragment in its structure as the host material of the light-emitting layer d34 may enhance the fluorescence luminescence properties of the light-emitting layer d34, and thereby enhance the light-emitting efficiency of the light-emitting layer d34. In addition, since all of the benzene rings in the conjugated fragment are of a π-π conjugated structure, and the electron cloud overlapping portion between molecules of the conjugated structure is large, which is favorable for the hopping transport of the electrons and holes between the molecules, the use of the material having the conjugated fragment in its structure as the host material of the light-emitting layer d34 is able to enhance the smoothness of transporting the electrons and holes in the light-emitting layer d34, i.e., enhance the electron mobility and hole mobility of the light-emitting layer d34, thereby promoting the formation of excitons in the light-emitting layer d34, and ultimately enhancing the light-emitting efficiency of the stacked light-emitting device D.

On this basis, in the stacked light-emitting device D provided in the embodiments of the present disclosure, the first host material may be selected as the host material of the N-type charge generation layer d41. In this case, the stacked light-emitting device D has at least two light-emitting units d30, and the light-emitting layer d34 of each light-emitting unit d30 uses the third host material as the host material of the light-emitting layer d34, therefore, the N-type charge generation layer d41 and the light-emitting layer d34 in the entire stacked light-emitting device D all have conjugated fragments (i.e., the stacked light-emitting device D includes at least three layers having conjugated fragments). Since the conjugated fragment contained in the structure of the first host material and the conjugated fragment contained in the structure of the third host material are both large conjugated rigid aromatic ring structures, the electron mobility of the N-type charge generation layer d41 and the light-emitting layer d34, as well as the fluorescent luminescent properties of the light-emitting layer d34, may be enhanced, and ultimately, the light-emitting efficiency of the stacked light-emitting device D may be extended by an even greater extent.

It will be noted that the present disclosure does not limit the number of the benzene rings contained in the above-described conjugated fragment, as long as all of the benzene rings contained therein are π-π conjugated structures.

In some examples, the above-described conjugated fragment has two benzene rings, and the two benzene rings are of a π-π conjugated structure. In this case, the conjugated fragment is a naphthalene fragment, its chemical formula is C₁₀H₈, and its structural formula is:

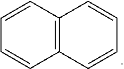

In some other examples, the above-described conjugated fragment has three benzene rings, and the three benzene rings are of a π-π conjugated structure. In this case, the conjugated fragment is an anthracene fragment, its chemical formula is C₁₄H₁₀, and its structural formula is:

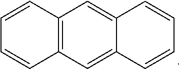 [SMILES: c1ccc2cc3ccccc3cc2c1]

In yet some other examples, the above-described conjugated fragment has five benzene rings, and the five benzene rings are of a π-π conjugated structure. In this case, the conjugated fragment is a pentacene fragment, its chemical formula is C₂₂H₁₄, and its structural formula is:

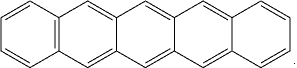 [SMILES: c1ccc2cc3cc4cc5ccccc5cc4cc3cc2c1]

In some embodiments, the above-described third host material has a structure shown in the following Formula (III):

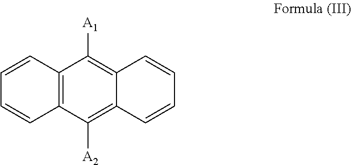

where A₁ and A₂ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted Cs to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group.

In the stacked light-emitting device D provided in the above embodiments, a material having an anthracene fragment (with the molecular formula of C₁₄H₁₀, and the structural formula of

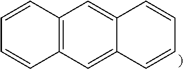 [SMILES: c1ccc2cc3ccccc3cc2c1]

is used as the host material of the light-emitting layer d34 (i.e., the third host material). On this basis, since the anthracene is of a large conjugated aromatic ring structure, which is characterized by high fluorescence quantum yields, adopting the material having the anthracene as the host material of the light-emitting layer d34 (i.e., the third host material) may enhance the fluorescence luminescence properties of the light-emitting layer d34, and thereby enhance the light-emitting efficiency of the light-emitting layer d34. In addition, since the electron cloud overlapping portion between molecules of the conjugated structure is large, which is favorable for the hopping-electron transport between molecules, the use of the material having the anthracene as the host material of the light-emitting layer d34 is able to enhance the smoothness of transporting the electrons in the light-emitting layer d34, i.e., enhance the electron mobility of the light-emitting layer d34, thereby promoting the formation of excitons in the light-emitting layer d34, and ultimately enhancing the light-emitting efficiency of the stacked light-emitting device D.

It will be noted that the embodiments of the present disclosure do not limit the method of obtaining the doped binary structure of the light-emitting layer d34. For example, the third guest material may be doped into the third host material by an ion implantation method or a diffusion method to obtain the doped binary structure.

In some embodiments, the third host material has a structure shown in any one of the following Formula (3-1) to Formula (3-12):

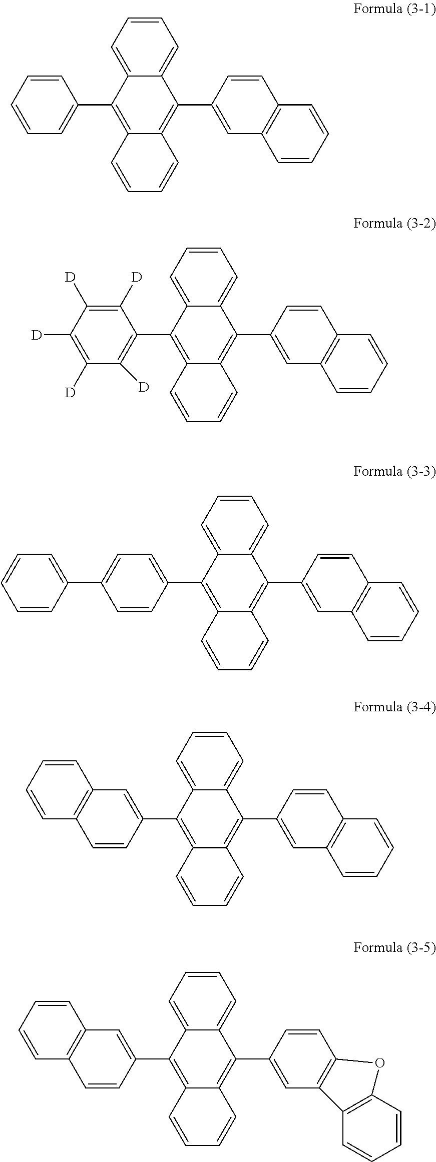

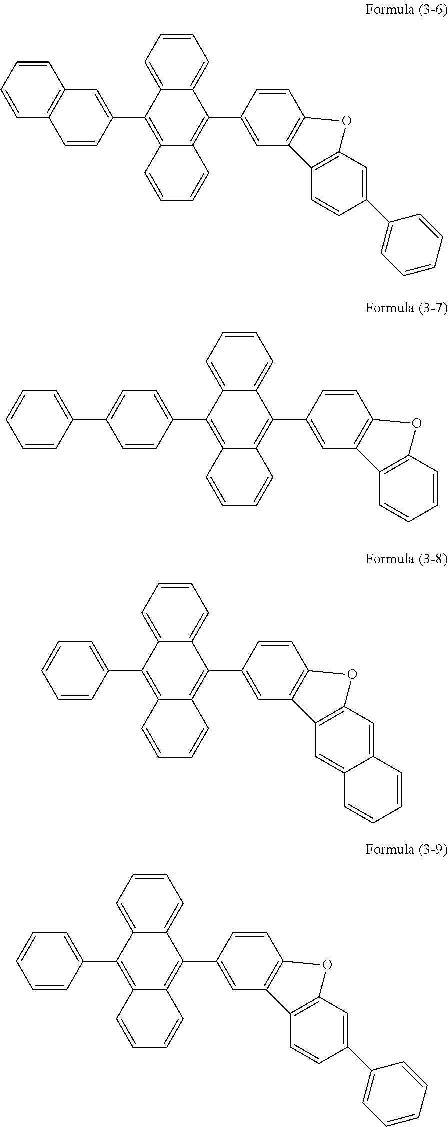 [SMILES: c1ccc(-c2ccc3c(c2)oc2ccc(-c4c5ccccc5c(-c5ccc6ccccc6c5)c5ccccc45)cc23)cc1 ; c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc3c(c2)oc2ccc(-c4c5ccccc5c(-c5ccccc5)c5ccccc45)cc23)cc1]

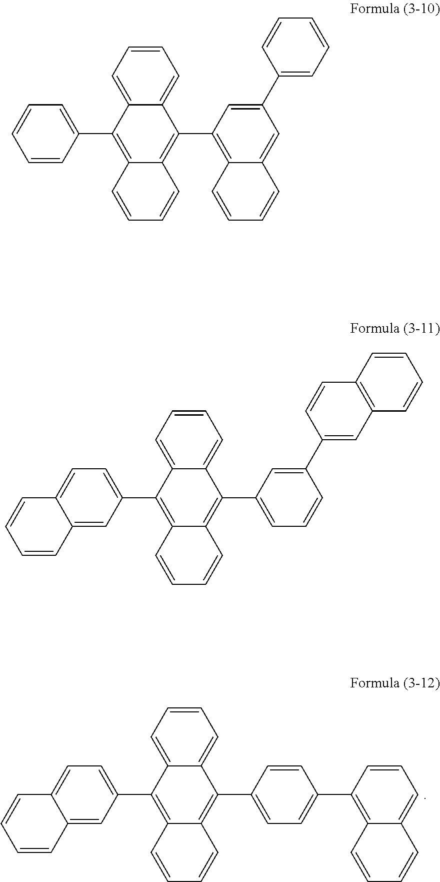

It will be noted that the materials shown in Formula (3-1) to Formula (3-12) above are only used as examples of the third host material, and any material satisfying Formula (III) above can be used as the third host material in the embodiments of the present disclosure.

In the above embodiments, in a case where the third host material has a structure as shown in any one of Formula (3-1) to Formula (3-12), the third host material has a relatively large number of aromatic ring structures, which is conducive to enhancing the fluorescent luminescent properties of the light-emitting layer d34.

In some examples, a method of preparing the material shown in Formula (3-2) above may include a step S21.

In S21, argon gas is introduced into a flask with a capacity of 500 mL; 3.2 g of raw material 4 with a structure shown in Formula E, 2.9 g of 2-bromonaphthalene, 0.23 g of tetrakis(triphenylphosphine)palladium (Pd(PPh₃)₄), 20 mL of 2M aqueous sodium carbonate (Na₂CO₃ aq), 20 mL of 1,2-dimethoxyethane, and 25 mL of toluene are added into the flask; the substances within the flask are heated to 150° C. with reflux reaction for 10 hours; the product obtained after the reflux reaction is cooled to room temperature, and then the product is filtered and precipitated as a solid; and the solid obtained by filtration is washed with water and methanol, and then the solid is recrystallized with toluene to obtain a material with a structure shown in Formula (3-2).

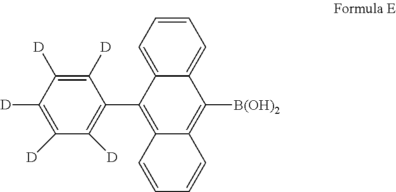 [SMILES: [2H]c1c([2H])c([2H])c(-c2c3ccccc3c(B(O)O)c3ccccc23)c([2H])c1[2H]]

Here, the reaction equation in the above S21 is as follows:

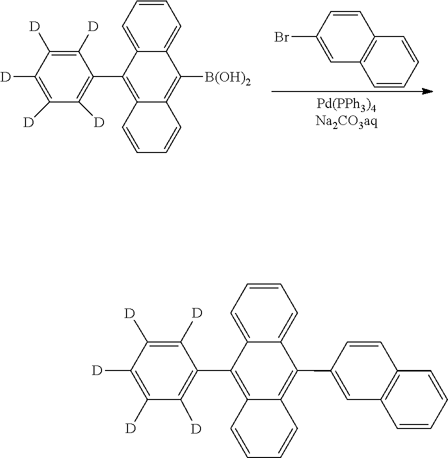 [SMILES: [2H]c1c([2H])c([2H])c(-c2c3ccccc3c(B(O)O)c3ccccc23)c([2H])c1[2H] ; Brc1ccc2ccccc2c1 ; [2H]c1c([2H])c([2H])c(-c2c3ccccc3c(-c3ccc4ccccc4c3)c3ccccc23)c([2H])c1[2H]]

It will be noted that since the materials shown in the above Formula (3-1) to Formula (3-12) have the same general formula, the methods of preparing the materials shown in Formula (3-1) as well as the materials shown in Formula (3-3) to Formula (3-12) are similar to that of the material shown in Formula (3-1), which will not be described herein.

In some examples, the above-described third guest material may be 4,4′-[1,4-phenylenedi-(1E)-2,1-ethenediyl]bis[N,N-diphenylbenzenamine](DSA-ph).

In a case where the light-emitting layer d34 of the stacked light-emitting device D is composed of the third host material and the third guest material provided in the above embodiments, it is favorable to enhance the electron mobility of the light-emitting layer d34, thereby increasing the probability of generating the excitons by electron-hole recombination in the light-emitting layer d34, and ultimately improving the light-emitting efficiency of the stacked light-emitting device D.

It will be noted that the embodiments of the present disclosure do not limit the materials constituting the first hole injection layer d311, the first hole transport layer d312, the first electron blocking layer d313, the first hole blocking layer d315, and the first electron transport layer d316 in the above-described first light-emitting unit d31.

In some examples, the material of the first hole injection layer d311 may be a doped substance of a hole transport material and an organic material having a strong electron-withdrawing group. Here, the hole transport material is an organic material having hole transport properties.

Among them, the hole transport material may be selected from an aromatic amine-like material, a dimethyl fluorene material, or a carbazole-like material that has hole transport properties. For example, the hole transport material may be selected from 4,4′-bis[N-(1-naphthyl)-N-phenylamino]biphenyl (NPB), N,N′-bis(3-methylphenyl)-N,N′-diphenyl-[1,1′-biphenyl]-4,4′-diamine (TPD), 4-phenyl-4′-(9-phenyl fluoren-9-yl)triphenylamine (BAFLP), 4,4′-bis[N-(9,9-dimethylfluoren-2-yl)-N-phenylamino]biphenyl (DFLDPBi), 4,4′-bis(9-carbazolyl)biphenyl (CBP), 9-phenyl-3-[4-(10-phenyl-9-anthryl)phenyl]-9H-carbazole (PCzPA), or 4,4′,4″-tris(N-3-methylphenyl-N-phenylamino)triphenylamine (m MTDATA).

An organic material having a strong electron-withdrawing group may be selected from hexacyano hexaazatriphenylene, 2,3,5,6-tetrafluoro-7,7,8,8-tetracyano-p-quinodimethane (F4TCNQ), or 1,2,3-tris[(cyano)(4-cyano-2,3,5,6-tetrafluorophenyl)methylene]cyclopropane.

It will be noted that at least one of the first hole transport layer d312 and the first electron blocking layer d313 may also be formed of the above hole transport material.

In some examples, both the above-described first hole blocking layer d315 and the first electron transport layer d316 may be formed of an aromatic heterocyclic compound. For example, the materials constituting the first hole blocking layer d315 and the first electron transport layer d316 may each be compounds with a nitrogen-containing six-membered ring structure, such as imidazole derivatives (such as benzimidazole derivatives, imidazopyridine derivatives, and benzo-imidazo-phenanthridine derivatives), zine derivatives (such as pyrimidine derivatives, and triazine derivatives), quinoline derivatives, isoquinoline derivatives, phenanthroline derivatives, or the like, or compounds having a phosphine oxide-based substituent on its heterocycle. For example, the material is 2-(4-biphenylyl)-5-(4-tert-butylphenyl)-1,3,4-oxadiazole (PBD), 1,3-bis[5-(para-tert-butylphenyl)-1,3,4-oxadiazol-2-yl]benzene (OXD-7), 3-(4-tert-butylphenyl)-4-phenyl-5-(4-biphenyl)-1,2,4-triazole (TAZ), 3-(4-tert-butylphenyl)-4-(4-ethylphenyl)-5-(4-biphenyl)-1,2,4-triazole (p-EtTAZ), bathophenanthroline (BPhen), 2,9-dimethyl-4,7-biphenyl-1,10-orthophenanthrolene (BCP), 4,4′-bis(5-methylbenzoxazol-2-yl)stilbene (BzOs), 1,3,5-tris(1-phenyl-1H-benzimidazol-2-yl)benzene (TPBi), or a substance doped with 8-hydroxyquinoline-lithium (Liq).

Similarly, the embodiments of the present disclosure do not limit the materials constituting the second hole transport layer d322, the second electron blocking layer d323, the second hole blocking layer d325, the second electron transport layer d326, and the second electron injection layer d327 in the above-described second light-emitting unit d32.

In some examples, the selection range of the materials constituting the second hole transport layer d322, the second electron blocking layer d323, the second hole blocking layer d325, and the second electron transport layer d326 may refer to the selection range of materials constituting the first hole transport layer d312, the first electron blocking layer d313, the first hole blocking layer d315, and the first electron transport layer d316 in the above-described embodiments respectively, which will not be described again here.

In some examples, the above-described second electron injection layer d327 may be formed of a metal (e.g., an alkali metal) or a metal-containing compound. For example, the material constituting the second electron injection layer d327 may be selected from lithium (Li), sodium (Na), potassium (K), rubidium (Rb), cesium (Cs), francium (Fr), magnesium (Mg), calcium (Ca), ytterbium (Yb), or lithium fluoride (LiF).

In summary, in the stacked light-emitting device D provided by the embodiments of the present disclosure, it is possible to ensure that each stacked connection layer d4 provides a stable number of carriers to the two light-emitting units d30 adjacent thereto, to enhance the electron mobility of the N-type charge generation layer d41 and the light-emitting layer d34, to increase the probability of generating the excitons by electron-hole recombination in the light-emitting layer d34, and to enhance the fluorescence luminescence properties of the light-emitting layer d34, thereby enhancing the light-emitting efficiency of the stacked light-emitting device D.

In order to objectively evaluate the technical effects of the embodiments of the present disclosure, the stacked light-emitting device D provided in the embodiments of the present disclosure will be exemplarily described by means of specific embodiments below.

### Embodiment 1

A stacked light-emitting device D is provided, which includes, as shown in FIG. 6, a first electrode d1, a first hole injection layer d311, a first hole transport layer d312, a first electron blocking layer d313, a first light-emitting layer d314, a first hole blocking layer d315, a first electron transport layer d316, an N-type charge generation layer d41, a P-type charge generation layer d42, a second hole transport layer d322, a second electron blocking layer d323, a second light-emitting layer d324, a second hole-blocking layer d325, a second electron transport layer d326, a second electron injection layer d327, and a second electrode d2.

In this embodiment, ITO is selected as a material constituting the first electrode d1; a mixture of m-MTDATA and F4TCNQ is selected as a material constituting the first hole injection layer d311; m-MTDATA is selected as a material constituting each of the first hole transport layer d312 and the second hole transport layer d322; CBP is selected as a material constituting each of the first electron blocking layer d313 and the second electron blocking layer d323; a material as shown in the above Formula (3-2) is selected as the third host material constituting the first light-emitting layer d314 and the second light-emitting layer d324, and a material shown in Formula (VI) is selected as the third guest material constituting the first light-emitting layer d314 and the second light-emitting layer d324; TPBi is selected as a material constituting each of the first hole blocking layer d315 and the second hole blocking layer d325; a mixture of BCP and Liq is selected as a material constituting each of the first electron transport layer d316 and the second electron transport layer d326; a material shown in the above Formula (1-1) is selected as the first host material constituting the N-type charge generation layer d41, and Yb is selected as the first guest material constituting the N-type charge generation layer d41; m-MTDATA is selected as the second host material constituting the P-type charge generation layer d42, and F4TCNQ is selected as the second guest material constituting the P-type charge generation layer d42; Yb is selected as a material constituting the second electron injection layer d327; and a mixture of Mg and Ag is selected as a material constituting the second electrode d2.

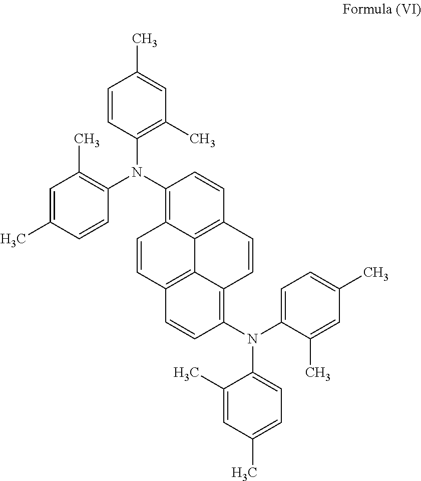

In the following, a method of preparing the above-described stacked light-emitting device D is described.

Taking the first electrode d1 as an anode as an example, a glass substrate may be coated with an ITO transparent conductive layer with a thickness of 150 nm, which serves as the first electrode d1. The glass substrate coated with the ITO transparent conductive layer is subjected to ultrasonication in a cleaning agent, and afterward, it is rinsed with deionized water. Then, the glass substrate with the ITO transparent conductive layer is subjected to ultrasonic degreasing in a solvent mixture of acetone and ethanol, and it is baked under a clean environment until water is completely removed, and then it is cleaned with ultraviolet light and ozone, and its surface is bombarded with a low-energy cation beam.

The obtained glass substrate described above is placed in a vacuum chamber, and the chamber is pumped to a vacuum (10-5 to 10-4 Pa); thereafter, the first hole injection layer d311 is formed on the first electrode d1 by vacuum evaporation. For example, the evaporation rate for the first hole injection layer d311 may be 0.1 nm/s, and the total evaporation film thickness may be 10 nm. Here, the first hole injection layer d311 is formed of the mixture of m-MTDATA and F4TCNQ, and the mass ratio of m-MTDATA to F4TCNQ is 97:3. The structural formula of m-MTDATA and the structural formula of F4TCNQ are shown as follows:

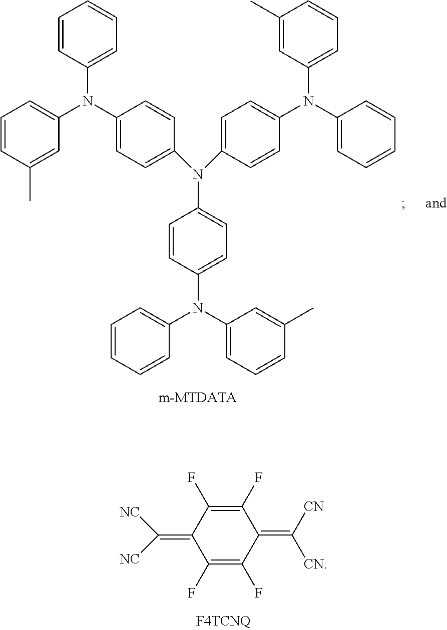 [SMILES: Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1 ; N#CC(C#N)=c1c(F)c(F)c(=C(C#N)C#N)c(F)c1F]

The first hole transport layer d312 is evaporated on the first hole injection layer d311. For example, the evaporation rate for the first hole transport layer d12 may be 0.1 nm/s, and the total evaporation film thickness may be 20 nm. Here, the first hole transport layer d312 is formed of the above-described m-MTDATA.

The first electron blocking layer d313 is evaporated on the first hole transport layer d312. For example, the evaporation rate for the first electron blocking layer d313 may be 0.1 nm/s, and the total evaporation film thickness may be 10 nm. Here, the first electron blocking layer d313 is formed of CBP, and the structural formula of CBP is shown as follows:

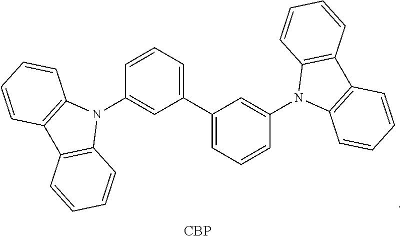 [SMILES: c1cc(-c2cccc(-n3c4ccccc4c4ccccc43)c2)cc(-n2c3ccccc3c3ccccc32)c1 ; "CBP"]

The first light-emitting layer d314 is evaporated on the first electron blocking layer d313. For example, the evaporation rate for the first light-emitting layer d314 may be 0.1 nm/s, and the total evaporation film thickness may be 20 nm. Here, the first light-emitting layer d314 is composed of a third host material and a third guest material, and the mass ratio of the third host material to the third guest material is 95:5. The third host material is a material shown in the above Formula (3-2), and the third guest material is a material shown in the above Formula (VI).

The first hole blocking layer d315 is evaporated on the first light-emitting layer d314. For example, the evaporation rate for the first hole blocking layer d315 may be 0.1 nm/s, and the total evaporation film thickness may be 5 nm. Here, the first hole blocking layer d315 is formed of TPBi, and the structural formula of TPBi is shown as follows:

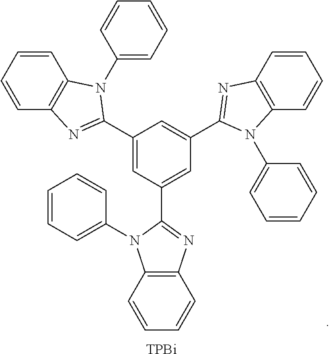

The first electron transport layer d316 is evaporated on the first hole blocking layer d315. For example, the evaporation rate for the first electron transport layer d316 may be 0.1 nm/s, and the total evaporation film thickness may be 30 nm. Here, the first electron transport layer d316 is formed of a mixture of BCP and Liq, with the mass ratio of BCP to Liq being 1:1. The structures of BCP and Liq are shown as follows:

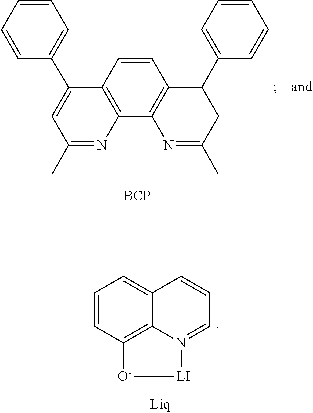

The N-type charge generation layer d41 is evaporated on the first electron transport layer d316. For example, the evaporation rate for the N-type charge generation layer d41 may be 0.1 nm/s, and the total evaporation film thickness may be 20 nm. Here, the N-type charge generation layer d41 is composed of a first host material and a first guest material, and the mass ratio of the first host material to the first guest material is 99:1. The first host material is a material shown in the above Formula (1-1), and the first guest material is Yb.

The P-type charge generation layer d42 is evaporated on the N-type charge generation layer d41. For example, the evaporation rate for the P-type charge generation layer d42 may be 0.1 nm/s, and the total evaporation film thickness may be 9 nm. Here, the P-type charge generation layer d42 is formed of a mixture of m-MTDATA and F4TCNQ, and the mass ratio of m-MTDATA to F4TCNQ is 95:5.

The second hole transport layer d322 is evaporated on the P-type charge generation layer d42. For example, the evaporation rate for the second hole transport layer d322 may be 0.1 nm/s, and the total evaporation film thickness may be 40 nm. Here, the second hole transport layer d322 is formed of m-MTDATA.

The methods of evaporating the second electron blocking layer d323 on the second hole transport layer d322, evaporating the second light-emitting layer d324 on the second electron blocking layer d323, and evaporating the second hole blocking layer d325 on the second light-emitting layer d324, and evaporating the second electron transport layer d326 on the second hole blocking layer d325 may refer to the above embodiments, which will not be described again here.

The second electron injection layer d327 is evaporated on the second electron transport layer d326. For example, the evaporation rate for the second electron injection layer d327 may be 0.1 nm/s, and the total evaporation film thickness may be 1 nm.

The second electrode d2 is evaporated on the second electron injection layer d327. For example, the evaporation rate for the second electrode d2 may be 0.1 nm/s, and the total evaporation film thickness may be 13 nm. Here, the second electrode d2 is formed of a mixture of Mg and Ag, and the mass ratio of Mg and Ag is 1:9.

After completing the above evaporation steps, resin is used as an encapsulating material, and ultraviolet light is used to cure the resin to encapsulate the above layers on the substrate to obtain the stacked light-emitting device D.

### Embodiment 2

In terms of the selection of materials for all layers constituting the stacked light-emitting device D, compared with Embodiment 1, the difference of this embodiment is that the material shown in the above Formula (3-8) is selected as the third host material constituting each of the first light-emitting layer d314 and the second light-emitting layer d324.

The method of manufacturing the stacked light-emitting device D in this embodiment can be referred to the above Embodiment 1 and will not be repeated herein.

### Embodiment 3

In terms of the selection of materials for all layers constituting the stacked light-emitting device D, compared with Embodiment 1, the difference of this embodiment is that the material as shown in the above Formula (1-6) is selected as the first host material constituting the N-type charge generation layer d41.

The method of manufacturing the stacked light-emitting device D in this embodiment can be referred to the above Embodiment 1 and will not be repeated herein.

### Embodiment 4

In terms of the selection of materials for all layers constituting the stacked light-emitting device D, compared with Embodiment 1, the difference of this embodiment is that the material as shown in the above Formula (3-8) is selected as the third host material constituting each of the first light-emitting layer d314 and the second light-emitting layer d324, and the material as shown in the above Formula (1-6) is selected as the first host material constituting the N-type charge generation layer d41.

The method of manufacturing the stacked light-emitting device D in this embodiment can be referred to the above Embodiment 1 and will not be repeated herein.

### Embodiment 5

In terms of the selection of materials for all layers constituting the stacked light-emitting device D, compared with Embodiment 1, the difference of this embodiment is that the material as shown in the above Formula (1-7) is selected as the first host material constituting the N-type charge generation layer d41.

The method of manufacturing the stacked light-emitting device D in this embodiment can be referred to the above Embodiment 1 and will not be repeated herein.

### Embodiment 6

In terms of the selection of materials for all layers constituting the stacked light-emitting device D, compared with Embodiment 1, the difference of this embodiment is that the material as shown in the above Formula (1-7) is selected as the first host material constituting the N-type charge generation layer d41, and the material as shown in the above Formula (3-8) is selected as the third host material constituting each of the first light-emitting layer d314 and the second light-emitting layer d324.

The method of manufacturing the stacked light-emitting device D in this embodiment can be referred to the above Embodiment 1 and will not be repeated herein.

### Comparison 1

In terms of the selection of materials for all layers constituting the comparative light-emitting device D_{ref}, compared with Embodiment 1, the difference of this comparison is that BCP is selected as the first host material constituting the N-type charge generation layer d41.

The method of manufacturing the comparative light-emitting device D_{ref} in this comparison can be referred to the above Embodiment 1 and will not be repeated herein.

### Comparison 2

In terms of the selection of materials for all layers constituting the comparative light-emitting device D_{ref}, compared with Embodiment 1, the difference of this comparison is that the material whose structure is shown in the following Formula (VII) is selected as the third host material constituting each of the first light-emitting layer d314 and the second light-emitting layer d324:

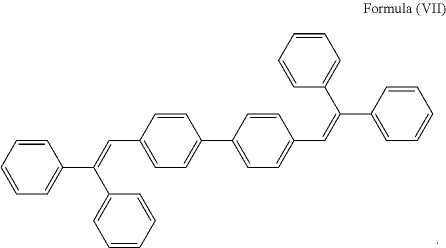 [SMILES: C(=C(c1ccccc1)c1ccccc1)c1ccc(-c2ccc(C=C(c3ccccc3)c3ccccc3)cc2)cc1]

The method of manufacturing the comparative light-emitting device D_{ref} in this comparison can be referred to the above Embodiment 1 and will not be repeated herein.

### Comparison 3

In terms of the selection of materials for all layers constituting the comparative light-emitting device D_{ref}, compared with Embodiment 1, the difference of this comparison is that BCP is selected as the first host material constituting the N-type charge generation layer d41, and the material whose structure is shown in the above Formula (VII) is selected as the third host material constituting each of the first light-emitting layer d314 and the second light-emitting layer d324.

Hereinafter, in conjunction with Table 1, performance comparisons are made between the stacked light-emitting device D provided in specific embodiments of the present disclosure (Embodiment 1 to Embodiment 6 described above) and the comparative light-emitting device D_{ref} provided in comparisons (Comparison 1 to Comparison 3 described above).

TABLE 1

| | N-CGL | EML host | Voltage | EQE | LT95 |
|---|---|---|---|---|---|
| Comparison 1 | BCP | Formula (3-2) | 100% | 100% | 100% |
| Comparison 2 | Formula (1-1) | Formula (VII) | 99% | 104% | 104% |
| Comparison 3 | BCP | Formula (VII) | 105% | 94% | 90% |
| Embodiment 1 | Formula (1-1) | Formula (3-2) | 98% | 108% | 126% |
| Embodiment 2 | Formula (1-1) | Formula (3-8) | 97% | 113% | 120% |
| Embodiment 3 | Formula (1-6) | Formula (3-2) | 95% | 111% | 130% |
| Embodiment 4 | Formula (1-6) | Formula (3-8) | 93% | 115% | 125% |
| Embodiment 5 | Formula (1-7) | Formula (3-2) | 92% | 116% | 132% |
| Embodiment 6 | Formula (1-7) | Formula (3-8) | 92% | 118% | 124% |

It will be noted that the percentage data of voltage, EQE, and LT95 in Table 1 are calculated based on the measured data of Comparison 1. That is, the specific value of the voltage measured in Comparison 1 is used as the denominator, and the specific value of the voltage measured in each comparison or each embodiment is used as the numerator, and the data of the voltage of each comparison or each embodiment in Table 1 is calculated; the specific value of the EQE measured in Comparison 1 is used as the denominator, and the specific value of the EQE measured in each comparison or each embodiment is used as the numerator, and the data of the EQE of each comparison or each embodiment in Table 1 is calculated; and the specific value of the LT95 measured in Comparison 1 is used as the denominator, and the specific value of the LT95 measured in each comparison or each embodiment is used as the numerator, and the data of the LT95 of each comparison or each embodiment in Table 1 is calculated.

Here, N-CGL represents the host material of the N-type charge generation layer d41 of the stacked light-emitting device D provided by the specific embodiments or by the comparative stacked light-emitting device D_{ref}; EML host represents the host material of the light-emitting layer in each light-emitting unit in the stacked light-emitting device D provided by the specific embodiments or by the comparative stacked light-emitting device D_{ref}; the voltage in Table 1 is a driving voltage of the stacked light-emitting device; EQE (external quantum efficiency) reflects the light-emitting efficiency of the stacked light-emitting device; and LT95 represents the time for the brightness of the stacked light-emitting device to drop to 95% of its initial brightness, which reflects the service life of the stacked light-emitting device.

According to the four sets of data of Comparison 1, Embodiment 1, Embodiment 3 and Embodiment 5 in Table 1, it can be seen that in a case where the third host material of the light-emitting layer d34 is the same, adopting the material provided by the embodiments of the present disclosure as the first host material of the N-type charge generation layer d41 may reduce the driving voltage of the stacked light-emitting device D, improve the light-emitting efficiency of the stacked light-emitting device D, and extend the service life of the stacked light-emitting device D.

According to the three sets of data of Comparison 2, Embodiment 1, and Embodiment 2 in Table 1, it can be seen that in a case where the first host material of the N-type charge generation layer d41 is the same, adopting the material provided by the embodiments of the present disclosure as the third host material of the light-emitting layer d34 may reduce the driving voltage of the stacked light-emitting device D, improve the light-emitting efficiency of the stacked light-emitting device D, and extend the service life of the stacked light-emitting device D.

According to the seven sets of data of Comparison 3, Embodiment 1, Embodiment 2, Embodiment 3, Embodiment 4, Embodiment 5, and Embodiment 6 in Table 1, it can be seen that adopting the material provided by the embodiments of the present disclosure as the first host material of the N-type charge generation layer d41, and adopting the material provided by the embodiments of the present disclosure as the third host material of the light-emitting layer d34 may significantly reduce the driving voltage of the stacked light-emitting device D, improve the light-emitting efficiency of the stacked light-emitting device D, and extend the service life of the stacked light-emitting device D.

Some embodiments of the present disclosure provide another light-emitting device D1. As shown in FIG. 7, the light-emitting device D1 includes a first electrode d1, a second electrode d2 and at least one light-emitting unit d30. The at least one light-emitting unit d30 is disposed between the first electrode d1 and the second electrode d2. The light-emitting unit d30 includes a light-emitting layer d34. Here, the light-emitting layer d34 has a doped binary structure including a third host material and a third guest material. A structure of the third host material has a conjugated fragment. The conjugated fragment has at least two benzene rings, and all of the benzene rings in the conjugated fragment are of a π-π conjugated structure.

In the light-emitting device D1 provided in the embodiments, a material having the conjugated fragment in its structure is selected as the host material of the light-emitting layer d34 (i.e., the third host material). On this basis, since the conjugated fragment has at least two benzene rings (aromatic rings), which is characterized by high fluorescence quantum yields, adopting the material having the conjugated fragment in its structure as the host material of the light-emitting layer d34 may enhance the fluorescence luminescence properties of the light-emitting layer d34, and thereby enhance the light-emitting efficiency of the light-emitting layer d34. In addition, since all of the benzene rings in the conjugated fragment are of a π-π conjugated structure, and the electron cloud overlapping portion between molecules of the conjugated structure is large, which is favorable for the hopping transport of the electrons and holes between the molecules, the use of the material having the conjugated fragment in its structure as the host material of the light-emitting layer d34 is able to enhance the smoothness of transporting the electrons and holes in the light-emitting layer d34, i.e., enhance the electron mobility and hole mobility of the light-emitting layer d34, thereby promoting the formation of excitons in the light-emitting layer d34, and ultimately enhancing the light-emitting efficiency of the stacked light-emitting device D.

It will be noted that the present disclosure does not limit the number of the benzene rings contained in the above-described conjugated fragment, as long as all of the benzene rings contained therein are π-π conjugated structures.

In some examples, the above-described conjugated fragment has two benzene rings, and the two benzene rings are of a π-π conjugated structure. In this case, the conjugated fragment is a naphthalene fragment, its chemical formula is C₁₀H₈, and its structural formula is:

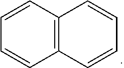

In some other examples, the above-described conjugated fragment has three benzene rings, and the three benzene rings are of a π-π conjugated structure. In this case, the conjugated fragment is an anthracene fragment, its chemical formula is C₁₄H₁₀, and its structural formula is:

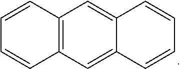 [SMILES: c1ccc2cc3ccccc3cc2c1]

In yet some other examples, the above-described conjugated fragment has five benzene rings, and the five benzene rings are of a π-π conjugated structure. In this case, the conjugated fragment is a pentacene fragment, its chemical formula is C₂₂H₁₄, and its structural formula is:

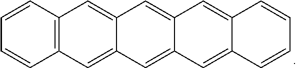 [SMILES: c1ccc2cc3cc4cc5ccccc5cc4cc3cc2c1]

In some embodiments, the above-described third host material has a structure shown in the following Formula (III):

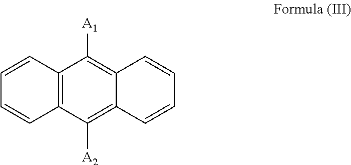

where A₁ and A₂ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group.

In the above embodiments, a material having an anthracene fragment (with the molecular formula of C₁₄H₁₀, and the structural formula of

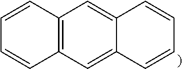 [SMILES: c1ccc2cc3ccccc3cc2c1]

is used as the host material of the light-emitting layer d34 (i.e., the third host material). On this basis, since the anthracene is of a large conjugated aromatic ring structure, which is characterized by high fluorescence quantum yields, adopting the material having the anthracene fragment as the host material of the light-emitting layer d34 may enhance the fluorescence luminescence properties of the light-emitting layer d34, and thereby enhance the light-emitting efficiency of the light-emitting layer d34. In addition, since the electron cloud overlapping portion between molecules of the conjugated structure is large, which is favorable for the hopping-electron transport between molecules, the use of the material having the anthracene fragment as the host material of the light-emitting layer d34 is able to enhance the smooth smoothness of transporting the electrons in the light-emitting layer d34, i.e., enhance the electron mobility of the light-emitting layer d34, thereby promoting the formation of excitons in the light-emitting layer d34, and ultimately enhancing the light-emitting efficiency of the light-emitting device D1.

It will be noted that the embodiments of the present disclosure do not limit the number of the light-emitting units d30 in the light-emitting device D1.

In some examples, as shown in FIG. 7, the above-described light-emitting device D1 includes only one light-emitting unit d33. In this way, the manufacturing cost of the light-emitting device D1 may be saved.

In this case, the light-emitting device D1 includes the first electrode d1, the light-emitting unit d33 and the second electrode d2 arranged along the direction Z away from the first electrode d1.

Here, the light-emitting unit d33 includes a first hole injection layer d3111, a first hole transport layer d3121, a first electron blocking layer d3131, a first light-emitting layer d3141, a first hole blocking layer d3151, a first electron transport layer d3161, and a first electron injection layer d3171 arranged along the direction Z away from the first electrode d1.

In some other embodiments, the above-described light-emitting device D1 includes two or more light-emitting units d30. In this case, the light-emitting device D1 is a stacked light-emitting device. In this way, the light-emitting efficiency of the light-emitting device D1 may be improved and the service life of the light-emitting device D1 may be extended.

In this case, taking the light-emitting device D1 including two light-emitting units d30 as an example, the structure of the light-emitting device D1 can be referred to FIG. 6.

The structure and material selection range of each layer in the above-described light-emitting unit d30 can refer to the above-described embodiments, which will not be described again here.

In some embodiments, the third host material has a structure shown in any one of the following Formula (3-1) to Formula (3-12):

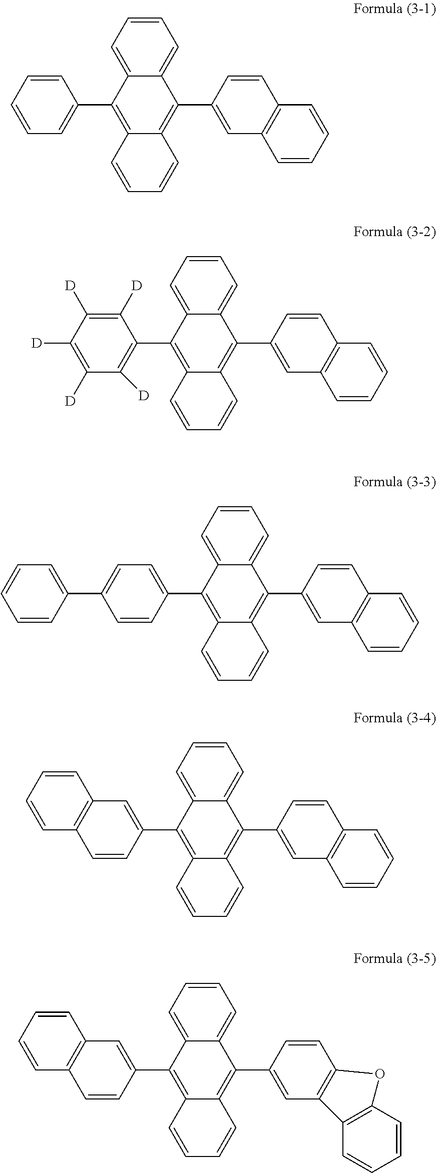

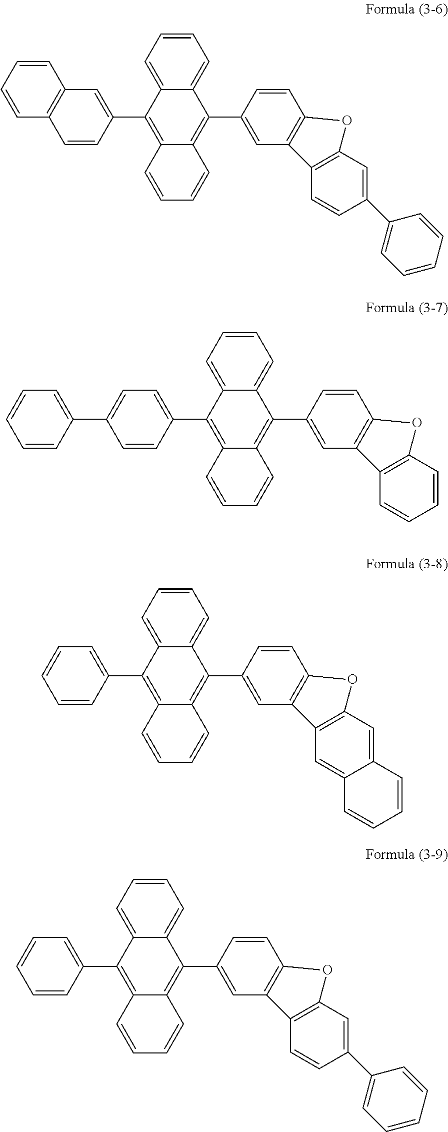 [SMILES: c1ccc(-c2ccc3c(c2)oc2ccc(-c4c5ccccc5c(-c5ccc6ccccc6c5)c5ccccc45)cc23)cc1 ; c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc3c(c2)oc2ccc(-c4c5ccccc5c(-c5ccccc5)c5ccccc45)cc23)cc1]

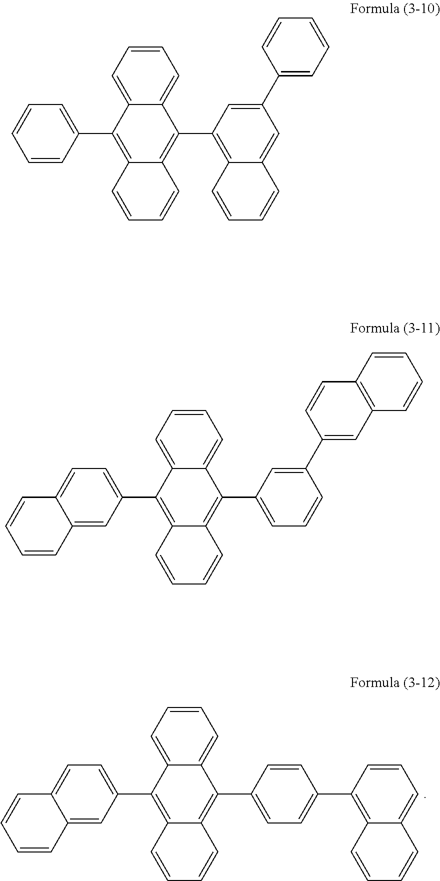

In the above embodiments, in a case where the third host material has a structure shown in any one of Formula (3-1) to Formula (3-12), the third host material has more aromatic ring structures, which is conducive to improving the fluorescence luminescence characteristics of the light-emitting layer d34.

In some examples, the light-emitting layer d34 has a doped binary structure including a third host material and a third guest material. For example, the above third guest material may be the above DSA-ph or the above material shown in the above Formula (VI).

In a case where the light-emitting layer d34 of the light-emitting device D1 is composed of the third host material and the third guest material provided in the above embodiments, it is beneficial to increase the electron mobility of the light-emitting layer d34, thereby increasing the probability of generating the excitons by electron-hole recombination in the light-emitting layer d34, and ultimately improving the light-emitting efficiency of the light-emitting device D1.

It will be noted that the embodiments of the present disclosure do not limit the method of obtaining the doped binary structure of the light-emitting layer d34. For example, the third guest material may be doped into the third host material by an ion implantation method or a diffusion method to obtain the doped binary structure.

Some embodiments of the present disclosure provide a display substrate 111. The display substrate 111 includes the substrate 1 as mentioned above, the circuit structure layer 2 disposed on the substrate 1, the light-emitting structure layer 3 disposed on the side of the circuit structure layer 2 away from the substrate 1, and the encapsulation layer 4 disposed on a side of the light-emitting structure layer 3 away from the substrate 1.

The circuit structure layer 2 includes a plurality of pixel driving circuits 10. The light-emitting structure layer 3 includes a plurality of light-emitting devices, one light-emitting device is connected to one pixel driving circuit 10. Here, at least one light-emitting device D is the stacked light-emitting device D as described in any of the above embodiments or the light-emitting device D1 as described in any of the above embodiments. The encapsulation layer 4 encapsulates the circuit structure layer 2 and the light-emitting structure layer 3 on the substrate 1.

Beneficial effects achieved by the display substrate 111 provided in the embodiments of the present disclosure are the same as the beneficial effects achieved by the stacked light-emitting device D or the light-emitting device D1 provided in any of the above embodiments, which will not be repeated here.

Some embodiments of the present disclosure provide a display apparatus 100, which includes the display substrate 111 as described in any of the above embodiments.

Beneficial effects achieved by the display apparatus 100 provided in the embodiments of the present disclosure are the same as the beneficial effects achieved by the display substrate 111 provided in any of the above embodiments, which will not be repeated here.

Some embodiments of the present disclosure provide an organic matter, which has a structure shown in the following Formula (III):

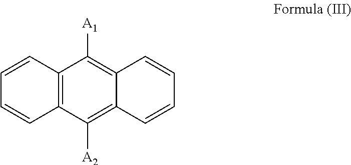

where A₁ and A₂ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group.

The organic matter provided in the embodiments of the present disclosure has an anthracene fragment (with the molecular formula of C₁₄H₁₀, and the structural formula of

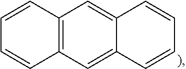 [SMILES: c1ccc2cc3ccccc3cc2c1]

and the substituents A₁ and A₂ on the anthracene fragment are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group, therefore, the fluorescence quantum yield of the organic matter may be improved, and the smoothness of transporting the electrons in the organic matter may be improved, thereby improving the electron mobility of the organic matter.

In some embodiments, the organic matter has a structure shown in any one of the following Formula (3-1) to Formula (3-12):

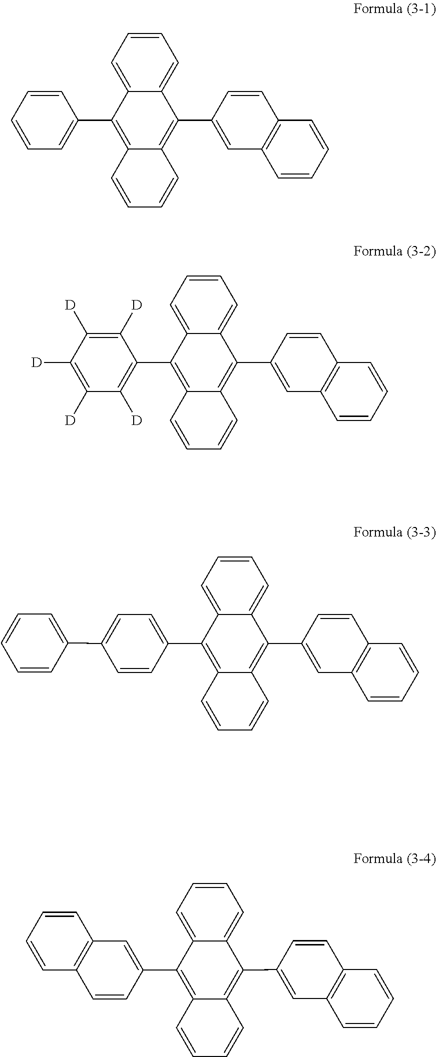

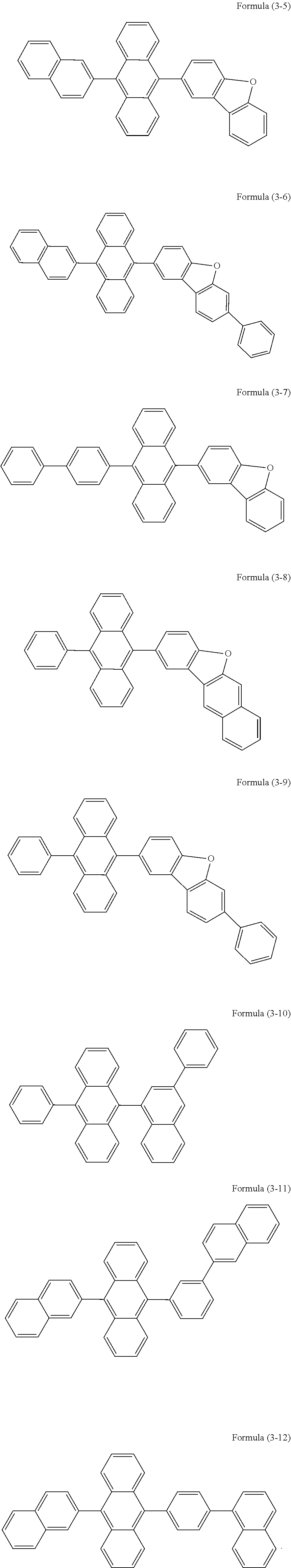

In the above embodiments, the organic matter has more aromatic ring structures, which is beneficial to further improving its fluorescence quantum yield.

To sum up, the organic matter provided in the embodiments of the present disclosure has an anthracene fragment (with the molecular formula of C₁₄H₁₀, and the structural formula of

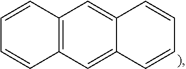 [SMILES: c1ccc2cc3ccccc3cc2c1]

and the substituents A₁ and A₂ on the anthracene fragment are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₆₀ aryloxy group, therefore, the fluorescence quantum yield and the electron mobility of the organic matter may be improved.

The foregoing description is only specific embodiments of the present disclosure, but the scope of protection of the present disclosure is not limited thereto. Any changes or replacements that a person skilled in the art could conceive of within the technical scope of the present disclosure shall be included in the protection scope of the present disclosure. Therefore, the protection scope of the present disclosure shall be subject to the protection scope of the claims.

1. A stacked light-emitting device, comprising:
a first electrode;
a second electrode;
at least two light-emitting units, stacked between the first electrode and the second electrode; and
at least one stacked connection layer, a stacked connection layer in the at least one stacked connection layer being disposed between every two adjacent light-emitting units in the at least two light-emitting units, and the stacked connection layer including an N-type charge generation layer and a P-type charge generation layer disposed in a stack, wherein the N-type charge generation layer is of a doped binary structure including a first host material and a first guest material; the P-type charge generation layer is of a doped binary structure including a second host material and a second guest material; an absolute value of a difference between a highest occupied molecular orbital energy level of the second host material and a highest occupied molecular orbital energy level of the first host material is greater than 0.3 electron volts; and an absolute value of a difference between a lowest unoccupied molecular orbital energy level of the second host material and a lowest unoccupied molecular orbital energy level of the first host material is greater than 0.1 electron volts.

2. The stacked light-emitting device according to claim 1, wherein the first guest material includes at least one of a metal or an organic matter, wherein
in a case where the first guest material is the metal, an absolute value of a difference between a work function of the first guest material and the lowest unoccupied molecular orbital energy level of the first host material is less than 1.0 electron volts;
in a case where the first guest material is the organic matter, an absolute value of a difference between a highest occupied molecular orbital energy level of the first guest material and the lowest unoccupied molecular orbital energy level of the first host material is less than 1.0 electron volts.

3. The stacked light-emitting device according to claim 1, wherein an absolute value of a difference between a lowest unoccupied molecular orbital energy level of the second guest material and the highest occupied molecular orbital energy level of the second host material is less than 0.5 electron volts.

4. The stacked light-emitting device according to claim 1, wherein a structure of the first host material has a conjugated fragment; and the conjugated fragment has at least two benzene rings, and all of the benzene rings in the conjugated fragment are of a π-π conjugated structure.

5. (canceled)

6. The stacked light-emitting device according to claim 1, wherein the first host material has a structure shown in formula (I): 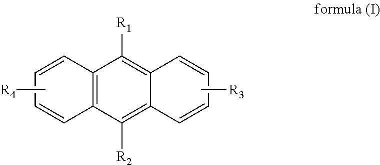
wherein R₁, R₂, R₃ and R₄ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, a substituted or unsubstituted C₆ to C₃₀ aryloxy group, and a structure shown in formula (II), wherein at least one of the R₁, the R₂, the R₃ and the R₄ has the structure shown in the formula (II): 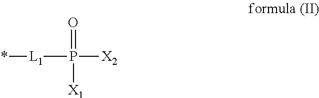
wherein * indicates a site connected to a carbon atom;
L₁ is selected from any one of: single bond, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₃₀ aryloxy group; and
X₁ are X₂ each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₃₀ aryloxy group.

7-9. (canceled)

10. The stacked light-emitting device according to claim 1, wherein the light-emitting units each include a light-emitting layer, and the light-emitting layer is of a doped binary structure including a third host material and a third guest material; a structure of the third host material has a conjugated fragment; and the conjugated fragment has at least two benzene rings, and all of the benzene rings in the conjugated fragment are of a π-π conjugated structure.

11. The stacked light-emitting device according to claim 10, wherein the third host material has a structure shown in formula (III): 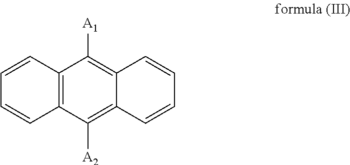
wherein A₁ and A₂ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₃₀ aryloxy group.

12. (canceled)

13. A stacked light-emitting device, comprising:
a first electrode;
a second electrode;
at least two light-emitting units, stacked between the first electrode and the second electrode; the light-emitting units each including a light-emitting layer; and
at least one stacked connection layer, a stacked connection layer in the at least one stacked connection layer being disposed between every two adjacent light-emitting units in the at least two light-emitting units, and the stacked connection layer including an N-type charge generation layer and a P-type charge generation layer disposed in a stack;
wherein at least three film layers in the stacked light-emitting device each include a material having a conjugated fragment, and the at least three film layers include at least one of the light-emitting layer and the N-type charge generation layer.

14. The stacked light-emitting device according to claim 13, wherein the conjugated fragment has at least two benzene rings, and all of the benzene rings in the conjugated fragment have a π-π conjugated structure; and/or
of the at least three film layers in the stacked light-emitting device, at least one material having a conjugated fragment has a phosphorus oxygen group in at least one substituent on the conjugated fragment; and/or
of the at least three film layers in the stacked light-emitting device, at least two film layers have different conjugated fragments.

15. (canceled)

16. (canceled)

17. The stacked light-emitting device according to claim 13, wherein the at least three film layers in the stacked light-emitting device include at least two light-emitting layers and an N-type charge generation layer located between the at least two light-emitting layers.

18. (canceled)

19. The stacked light-emitting device according to claim 13, wherein the light-emitting layer includes a doped binary structure including a third host material and a third guest material; and a structure of the third host material has the conjugated fragment.

20. The stacked light-emitting device according to claim 19, wherein the third host material has a structure shown in formula (III): 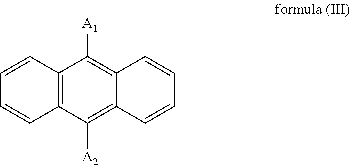
wherein A₁ and A₂ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₃₀ aryloxy group.

21. (canceled)

22. The stacked light-emitting device according to claim 13, wherein the N-type charge generation layer includes a doped binary structure including a first host material and a first guest material; and a structure of the first host material has the conjugated fragment.

23. The stacked light-emitting device according to claim 22, wherein the P-type charge generation layer is of a doped binary structure including a second host material and a second guest material, wherein
an absolute value of a difference between a highest occupied molecular orbital energy level of the second host material and a highest occupied molecular orbital energy level of the first host material is greater than 0.3 electron volts; and an absolute value of a difference between a lowest unoccupied molecular orbital energy level of the second host material and a lowest unoccupied molecular orbital energy level of the first host material is greater than 0.1 electron volts.

24. The stacked light-emitting device according to claim 22, wherein the first guest material includes at least one of a metal or an organic matter, wherein
in a case where the first guest material includes the metal, an absolute value of a difference between a work function of the metal included in the first guest material and the lowest unoccupied molecular orbital energy level of the first host material is less than 1.0 electron volts;
in a case where the first guest material includes the organic matter, an absolute value of a difference between a highest occupied molecular orbital energy level of the organic matter included in the first guest material and the lowest unoccupied molecular orbital energy level of the first host material is less than 1.0 electron volts.

25. The stacked light-emitting device according to claim 23, wherein an absolute value of a difference between a lowest unoccupied molecular orbital energy level of the second guest material and the highest occupied molecular orbital energy level of the second host material is less than 0.5 electron volts.

26. The stacked light-emitting device according to claim 22, wherein the first host material has a structure shown in formula (I): 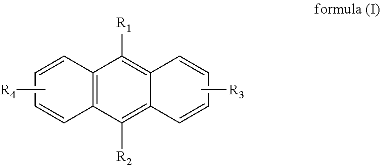
wherein R₁, R₂, R₃ and R₄ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, a substituted or unsubstituted C₆ to C₃₀ aryloxy group, and a structure shown in formula (II), wherein at least one of the R₁, the R₂, the R₃ and the R₄ has the structure shown in the formula (II): 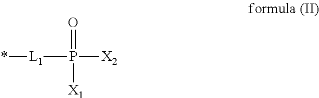
wherein * indicates a site connected to a carbon atom;
L₁ is selected from any one of: single bond, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₃₀ aryloxy group; and
X₁ are X₂ each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted C₁ to C₂₀ alkoxy group, and a substituted or unsubstituted C₆ to C₃₀ aryloxy group.

27. (canceled)

28. A light-emitting device, comprising:
a first electrode;
a second electrode; and
at least one light-emitting unit, disposed between the first electrode and the second electrode; the light-emitting units each including a light-emitting layer;
wherein the light-emitting layer is of a doped binary structure including a third host material and a third guest material; a structure of the third host material has a conjugated fragment; and the conjugated fragment has at least two benzene rings, and all of the benzene rings in the conjugated fragment are of a π-π conjugated structure.

29. The light-emitting device according to claim 28, wherein the third host material has a structure shown in formula (III): 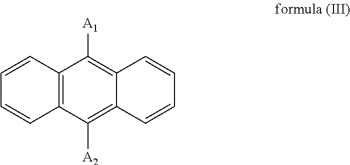
wherein A₁ and A₂ are each independently selected from any one of: hydrogen, deuterium, halogen, a substituted or unsubstituted C₆ to C₆₀ aryl group, a substituted or unsubstituted C₆ to C₆₀ hetero-aryl group, a substituted or unsubstituted C₁ to C₂₀ alkyl group, a substituted or unsubstituted C₃ to C₂₀ cycloalkyl group, a substituted or unsubstituted C₁ to C₂₀ hetero-alkyl group, a substituted or unsubstituted C₇ to C₃₀ aralkyl group, a substituted or unsubstituted Cᵢ to C₂₀ alkoxy group, and a substituted or unsubstituted C₆ to C₃₀ aryloxy group.

30. (canceled)

31. A display substrate, comprising:
a substrate;
a plurality of light-emitting devices, at least one of the plurality of light-emitting devices being the stacked light-emitting device according to claim 1;
a plurality of pixel driving circuits for driving the plurality of light-emitting devices to emit light; and
an encapsulation layer, for encapsulating the plurality of light-emitting devices and the plurality of pixel driving circuits.

32-34. (canceled)
